(19) United States
(12) Patent Application Publication
(10) Pub. No.: US 2023/0064976 A1
(43) Pub. Date: Mar. 2, 2023

(54) AMORPHOUS FORM OR CRYSTALLINE FORM OF 2-INDOLINOLINOLOLYLSPIRONONE COMPOUNDS OR THEIR SALTS, SOLVENT COMPLEXES

(71) Applicants: ASCENTAGE PHARMA (SUZHOU) CO., LTD., Suzhou, Jiangsu (CN); ASCENTAGE PHARMA GROUP CORP LIMITED, Hong Kong (CN)

(72) Inventors: Jianfeng WEN, Jiangsu (CN); Jianpeng FENG, Jiangsu (CN); Tianzhu WU, Jiangsu (CN); Weidong LI, Jiangsu (CN); Yanqiong LIN, Jiangsu (CN); Zongbin LI, Jiangsu (CN)

(73) Assignees: ASCENTAGE PHARMA (SUZHOU) CO., LTD., Jiangsu (CN); ASCENTAGE PHARMA GROUP CORP LIMITED, Hong Kong (CN)

(21) Appl. No.: 17/794,472

(22) PCT Filed: Jan. 22, 2021
(86) PCT No.: PCT/CN2021/073236

§ 371 Date: Jul. 21, 2022

(30) Foreign Application Priority Data
Jan. 23, 2020 (CN) 202010076778.6
Jan. 15, 2021 (CN) 202110051746.5

(51) Int. Cl. C07D 487/10

(52) U.S. Cl. CPC C07D 487/10; C07B 2200/13

(57) ABSTRACT

Amorphous form or crystalline form of a 2-indolinolinololylspironone compound or its salt and solvate used as an MDM2 inhibitor, a preparation method and an application thereof. The amorphous form or crystalline form of the invention has good stability and is of great value for drug development, preparation development and production.

### TECHNICAL FIELD

The invention belongs to the field of medicinal chemistry, in particular to an amorphous form or crystalline form of a 2-Indolinolinololylspironone compound or its salt and solvent complex used as an MDM2 inhibitor and its preparation method and application.

### BACKGROUND ART

The p53 tumor suppressor plays an important role in controlling cell cycle progression, senescence, and apoptosis (Vogelstein et al., Nature 408:307 (2000); Goberdhan, CancerCell 7:505 (2005)). MDM2 and p53 are part of an autoregulatory feedback loop (Wu et al., GenesDev. 7:1126 (1993)). MDM2 is transcriptionally activated by p53 and MDM2, which in turn represses p53 activity through at least three mechanisms (Wu et al., GenesDev. 7:1126 (1993)). First, the MDM2 protein directly binds to the p53 transactivation domain, and consequently inhibits p53-mediated transactivation; second, the MDM2 protein contains a nuclear export signal sequence, and when bound to p53, induces the nuclear export of p53, thereby preventing p53 binding to the targeted DNA; and third, the MDM2 protein is an E3 ubiquitin ligase and, when bound to p53, is able to promote p53 degradation.

WO2015/161032A1 disclosed a 2-Indolinolinololylspironone compound that inhibits MDM2-P53 interaction and therefore activates the function of p53 and p53-related proteins for therapeutic applications, which not only showed improved stability of their chemical solution, but also showed unexpectedly improved antitumor activity, including complete tumor regression in animal models of human osteosarcoma. Specifically, Compound No. 8 (referred to herein as Compound 1), described in its labeling, binds to MDM2 protein with IC50 values and Ki values of 3.8 nM and <1.0 nM, respectively. The compound blocks the interaction of MDM2 with P53 and induces periodic arrest and apoptosis in a P53 dependent manner with the structural formula:

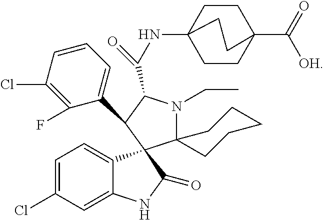

However, the current literature including this patent application mainly reports the structure and pharmacological activity of this type of compound, and has not conducted any research and report on its polymorphism, amorphous and other forms.

Due to the influence of various factors such as the configuration, conformation, molecular arrangement, molecular force, eutectic substance, etc. of the molecular structure of a solid substance, the spatial arrangement of the molecular lattice is different, forming two or more different crystal structures, this phenomenon is called “Polymorphism Phenomenon” or “Phenomenon”. “Polymorphism” is widespread in solid drugs, and there may be differences in physical and chemical properties between different crystal forms of the same drug, such as appearance, density, hardness, melting point, solubility, stability, dissolution rate, dissolution rate, bioavailability, etc. There may be significant differences, and this phenomenon is particularly obvious in oral solid preparations. In addition, the existence and quantity of polymorphic compounds are unpredictable. Different crystalline forms of the same drug have significant differences in solubility, melting point, density, stability, etc., which affect the uniformity, bioavailability, efficacy and safety.

In addition to polymorphs, some solid compounds may also exist in amorphous forms. Amorphous refers to the structure of some imperfectly crystalline amorphous regions (amorphous regions) or the formation of some amorphous solids (non-crystalline). For a specific solid drug, its amorphous form and quantity are also unpredictable, and may also have a significant impact on the solubility, melting point, density, and stability of the drug.

Therefore, in the development of new drugs, a comprehensive screening of crystalline and amorphous forms of drug compounds is required, considering multiple factors. In particular, for the above-mentioned compound 1 used as an MDM2 inhibitor, the development of an amorphous or crystalline form of the compound or its salt or solvate that may have pharmaceutical value can improve the stability, solubility, and bioavailability of the compound. It has potential medicinal and clinical value.

### TECHNICAL FIELD

The present invention provides amorphous or crystalline forms of 2-Indolinolinololylspironone compounds or their salts and solvates used as MDM2 inhibitors, as well as preparation methods and applications thereof. The amorphous form or crystalline form of the present invention has good stability and is of great value for drug development, formulation development and production.

In the following description, certain specific details are set forth in order to provide a thorough understanding of various embodiments of the present invention. However, those skilled in the art will understand that the present invention can be practiced without these details. The following description of several embodiments is made with the understanding that the present disclosure is regarded as an example of the claimed subject matter, and is not intended to limit the appended claims to the specific embodiments shown. The headings used throughout the present invention are provided for convenience only and should not be construed as limiting the claims in any way. The embodiment shown under any heading can be combined with the embodiment shown under any other heading.

In addition, when referring to, for example, XRPD diagrams, DSC diagrams, TGA diagrams, DSC diagrams, etc., the term “substantially as shown” means that it is not necessarily the same as those described herein, but when considered by a person of ordinary skill in the art, the spectrum falls within the limits of experimental error or deviation.

First, the present invention provides the amorphous or crystalline form of the compound 1 below, or a salt or solvate thereof:

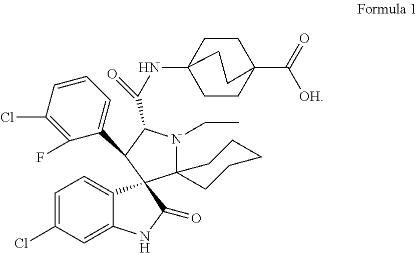

The chemical name of the compound is
4-((3′R,4'S,5′R)-6″-Chloro-4′-(3-chloro-2-fluorophenyl)-1′-ethyl-2″-oxodispiro[cyclohexane-1,2′-pyrrolidine-3′,3″-indoline]-5′-carboxamido) bicyclo[2.2.2]octane-1-carboxylic acid, CAS number is 1818393-16-6.

Specifically, the form may be the following specific forms:

### 1) The Compound 1 Sulfate Salt Amorphous Form I

In one embodiment, the form is the compound 1 sulfate salt amorphous form I. In one embodiment, it has:
1) Basically the X-ray diffraction (XRD) diagram as shown in FIG. 1;
2) Basically the Thermal Gravimetric Analysis (TGA) diagram as shown in FIG. 2;
3) Basically the Differential Scanning calorimetry (DSC) diagram as shown in FIG. 3;
4) Basically the Dynamic Vapour Sorption (DVS) diagram as shown in FIG. 4; and/or
5) Basically the adsorption isotherm curve as shown in FIG. 5.

### 2) The Compound 1 Hydrochloride Amorphous Form II

In one embodiment, the form is the compound 1 hydrochloride amorphous form II. In one embodiment, it has basically the X-ray diffraction (XRD) diagram as shown in FIG. 6.

### 3) The Compound 1 Hydrochloride Crystalline Form III

In one embodiment, the form is the compound 1 hydrochloride amorphous form III. In one embodiment, it has:
1) Basically the X-ray diffraction (XRD) diagram as shown in FIG. 7;
2) Basically the Thermal Gravimetric Analysis (TGA) diagram as shown in FIG. 8;
3) Basically the Differential Scanning calorimetry (DSC) diagram as shown in FIG. 9.

### 4) The Compound 1 Hydrochloride Crystalline Form IV

In one embodiment, the form is the compound 1 hydrochloride amorphous form IV. In one embodiment, it has basically the X-ray diffraction (XRD) diagram as shown in FIG. 10.

### 5) The Compound 1 Maleate Crystalline Form V

In one embodiment, the form is the compound 1 maleate crystalline form V. In one embodiment, it has:
1) Basically the X-ray diffraction (XRD) diagram as shown in FIG. 11;
2) Basically the Thermal Gravimetric Analysis (TGA) diagram as shown in FIG. 12;
3) Basically the Differential Scanning calorimetry (DSC) diagram as shown in FIG. 13;
4) Basically the Dynamic Vapour Sorption (DVS) diagram as shown in FIG. 14.

### 6) The Compound 1 Hydrobromide Crystalline Form VI

In one embodiment, the form is the compound 1 hydrobromide crystalline form VI. In one embodiment, it has:
1) Basically the X-ray diffraction (XRD) diagram as shown in FIG. 15;
2) Basically the Thermal Gravimetric Analysis (TGA) diagram as shown in FIG. 16;
3) Basically the Differential Scanning calorimetry (DSC) diagram as shown in FIG. 17;
4) Basically the Dynamic Vapour Sorption (DVS) diagram as shown in FIG. 18.

### 7) The Compound 1 Mesylate Amorphous Form VII

In one embodiment, the form is the compound 1 mesylate amorphous form VII. In one embodiment, it has basically the X-ray diffraction (XRD) diagram as shown in FIG. 19.

### 8) The Compound 1 Sodium Salt Amorphous Form VIII

In one embodiment, the form is the compound 1 sodium salt amorphous form VIII. In one embodiment, it has:
1) Basically the X-ray diffraction (XRD) diagram as shown in FIG. 20;
2) Basically the Thermal Gravimetric Analysis (TGA) diagram as shown in FIG. 21;
3) Basically the Differential Scanning calorimetry (DSC) diagram as shown in FIG. 22;
4) Basically the Dynamic Vapour Sorption (DVS) diagram as shown in FIG. 23.

### 9) The Compound 1 Potassium Salt Amorphous Form IX

In one embodiment, the form is the compound 1 potassium salt amorphous form IX. In one embodiment, it has:
1) Basically the X-ray diffraction (XRD) diagram as shown in FIG. 24;
2) Basically the Thermal Gravimetric Analysis (TGA) diagram as shown in FIG. 25;
3) Basically the Differential Scanning calorimetry (DSC) diagram as shown in FIG. 26;
4) Basically the Dynamic Vapour Sorption (DVS) diagram as shown in FIG. 27.

### 10) The Compound 1 Crystalline Form X

In one embodiment, the form is the crystalline form X of the compound 1, which has characteristic peaks at the following positions in the XRPD diagram represented by 2θ angles: 9.080±0.2°, 13.820±0.2°, 14.262±0.2°, 15.543±0.2° and 19.160±0.2°.
In a preferred embodiment, the crystalline form X of the compound 1 has XRPD characteristic peaks at positions substantially as shown in Table 1 below and/or an XRPD pattern substantially as shown in FIG. 28.

TABLE 1

| (2θ°) ± 0.2° | I(%) | d (A) |
|---|---|---|
| 6.381 | 14.1 | 13.8396 |
| 8.180 | 1.2 | 10.8004 |
| 9.080 | 100.0 | 9.7316 |
| 9.741 | 18.5 | 9.0726 |
| 10.438 | 3.7 | 8.4682 |
| 10.744 | 2.2 | 8.2272 |
| 11.043 | 12.0 | 8.0053 |
| 11.537 | 5.2 | 7.6639 |
| 11.938 | 8.7 | 7.4070 |
| 12.658 | 6.9 | 6.9873 |
| 13.820 | 56.6 | 6.4025 |
| 14.262 | 61.1 | 6.2049 |
| 14.977 | 14.6 | 5.9105 |
| 15.220 | 9.2 | 5.8166 |
| 15.543 | 44.8 | 5.6962 |
| 16.047 | 7.6 | 5.5184 |
| 16.919 | 17.2 | 5.2362 |
| 17.203 | 8.4 | 5.1503 |
| 17.480 | 11.3 | 5.0693 |
| 17.701 | 7.9 | 5.0064 |
| 18.099 | 5.9 | 4.8972 |
| 18.456 | 5.2 | 4.8033 |
| 18.808 | 19.5 | 4.7143 |
| 19.160 | 30.8 | 4.6283 |
| 19.438 | 0.7 | 4.5628 |

In some preferred embodiments, the crystalline form X of the compound 1 further has one or more of the following characteristics:
1) In the TGA diagram, there is a weight loss of 2.5±0.5% by weight between 10-150° C., and the decomposition temperature is 260±10° C.;
2) In the DSC diagram, there are two small absorption peaks near 193° C. and 211° C.; and/or
3) In the DVS diagram, 2±0.5% of the surface solvent is lost after the DVS ends, 0% RH-60% RH water absorption <0.1% (almost no water absorption), 60% RH-80% RH weight change is 1.6±0.2% (Slightly hygroscopic).
In some preferred embodiments, the crystalline form X of the compound 1 further has one or more of the following characteristics:
1) Basically the TGA diagram as shown in FIG. 29;
2) Basically the DSC diagram as shown in FIG. 30; and/or
3) Basically the DVS diagram as shown in FIG. 31.

### 11) The Compound 1 Monohydrate Crystalline Form XI

In one embodiment, the form is the crystalline form XI of the compound 1 monohydrate, which has characteristic peaks at the following positions in the XRPD diagram represented by 2θ angles: 6.999±0.2°, 11.319±0.2°, 11.522±0.2° and 17.485±0.2°.

In a preferred embodiment, the monohydrate crystalline form XI of the compound 1 has characteristic peaks at the following positions in the XRPD diagram represented by 2θ angles: 6.999±0.2°, 9.858±0.2°, 11.319±0.2°, 11.522±0.2°, 12.341±0.2°, 13.282±0.2°, 17.485±0.2°, 17.923±0.2°, 19.159±0.2° and 28.644±0.2°.

In a preferred embodiment, the monohydrate crystalline form XI of the compound 1 has XRPD characteristic peaks at positions substantially as shown in Table 2 below and/or an XRPD pattern substantially as shown in FIG. 32.

TABLE 2

| (2θ°) ± 0.2° | I(%) | d (A) |
|---|---|---|
| 6.999 | 100.0 | 12.6188 |
| 7.480 | 18.7 | 11.8086 |
| 9.858 | 32.9 | 8.9651 |
| 11.319 | 64.9 | 7.8106 |
| 11.522 | 77.6 | 7.6736 |
| 11.824 | 17.2 | 7.4787 |
| 12.341 | 29.2 | 7.1661 |
| 13.282 | 38.1 | 6.6604 |
| 13.602 | 15.2 | 6.5047 |
| 14.890 | 2.6 | 5.9445 |
| 15.383 | 10.1 | 5.7554 |
| 16.043 | 16.8 | 5.5198 |
| 16.421 | 33.4 | 5.3937 |
| 16.702 | 7.0 | 5.3037 |
| 17.485 | 46.8 | 5.0680 |
| 17.923 | 38.7 | 4.9450 |
| 18.383 | 31.6 | 4.8222 |
| 19.159 | 37.8 | 4.6287 |
| 19.721 | 24.5 | 4.4980 |
| 20.042 | 14.0 | 4.4266 |
| 20.762 | 33.7 | 4.2747 |
| 21.222 | 15.1 | 4.1831 |
| 22.084 | 9.2 | 4.0218 |
| 22.920 | 1.8 | 3.8770 |
| 23.723 | 30.6 | 3.7475 |

In some preferred embodiments, the crystalline form XI of the compound 1 monohydrate also has one or more of the following characteristics:

1) In the TGA diagram, there is a weight loss of 2.4±0.5% by weight before 100° C., which is about one water molecule, and the decomposition temperature is 262±2° C.;

2) In the DSC diagram, there is a broad endothermic peak at 90° C.-140° C., the melting point of the sample is 243±3° C., and it decomposes after melting; and/or

3) In the DVS diagram, the weight change of 0% RH-80% RH is 0.17±0.05% (non-hygroscopic).

In some preferred embodiments, the crystalline form XI of the compound 1 monohydrate also has one or more of the following characteristics:

1) Basically the TGA diagram as shown in FIG. 33;

2) Basically the DSC diagram as shown in FIG. 34; and/or

3) Basically the DVS diagram as shown in FIG. 35.

12) The Compound 1 Di-Trifluoroethanol Solvate Crystal Form XII

In one embodiment, the form is the di-trifluoroethanol solvate crystalline form XII of the compound 1, which has characteristic peaks at the following positions in the XRPD diagram represented by 2θ angles: 6.601±0.2°, 11.482±0.2°, 15.219±0.2°, 17.283±0.2°, 19.826±0.2° and 22.862±0.2°.

In a preferred embodiment, the di-trifluoroethanol solvate crystalline form XII of the compound 1 has XRPD characteristic peaks at positions substantially as shown in Table 3 below and/or an XRPD pattern substantially as shown in FIG. 36.

TABLE 3

| (2θ°) ± 0.2° | I(%) | d (A) |
|---|---|---|
| 5.800 | 4.1 | 15.2254 |
| 6.601 | 18.7 | 13.3791 |
| 9.233 | 3.4 | 9.5701 |
| 9.496 | 3.4 | 9.3060 |
| 10.713 | 8.3 | 8.2510 |
| 11.482 | 17.9 | 7.7004 |
| 11.860 | 8.9 | 7.4560 |
| 12.344 | 5.7 | 7.1643 |
| 13.161 | 3.5 | 6.7216 |
| 15.018 | 6.7 | 5.8944 |
| 15.219 | 17.1 | 5.8170 |
| 15.560 | 8.9 | 5.6902 |
| 16.465 | 4.0 | 5.3794 |
| 16.917 | 7.0 | 5.2366 |
| 17.283 | 100.0 | 5.1267 |
| 17.764 | 15.9 | 4.9889 |
| 18.014 | 3.6 | 4.9202 |
| 18.357 | 2.0 | 4.8290 |
| 19.081 | 11.0 | 4.6473 |
| 19.826 | 64.6 | 4.4745 |
| 20.243 | 17.3 | 4.3831 |
| 20.694 | 7.6 | 4.2887 |
| 20.925 | 16.8 | 4.2419 |

In some preferred embodiments, the di-trifluoroethanol solvate crystalline form XII of the compound 1 further has one or more of the following characteristics:

1) In the TGA diagram, there is a weight loss of 27.7±1.0% by weight before 150° C., which is about two trifluoroethanol molecules, and the decomposition temperature is 264±2° C.; and/or

2) In the DSC diagram, there is a broad endothermic peak at 45° C.-150° C., which is caused by the removal of trifluoroethanol molecules.

In some preferred embodiments, the ditrifluoroethanol solvate crystalline form XII of the compound 1 further has one or more of the following characteristics:

1) Basically the TGA diagram as shown in FIG. 37; and/or

2) Basically the DSC diagram as shown in FIG. 38.

13) The Compound 1 Semi-Dimethyl Sulfoxide Solvate Crystal Form XIII

In one embodiment, the form is the semi-dimethyl sulfoxide solvate crystalline form XIII of the compound 1, which has characteristic peaks at the following positions in the XRPD diagram represented by 2θ angles: 6.737±0.2°, 9.302±0.2°, 9.494±0.2°, 15.957±0.2°, 17.240±0.2°, 17.683±0.2°, 18.520±0.2° and 19.946±0.2°.

In a preferred embodiment, the semi-dimethyl sulfoxide solvate crystalline form XIII of the compound 1 has XRPD characteristic peaks at positions substantially as shown in Table 4 below and/or an XRPD pattern substantially as shown in FIG. 39.

TABLE 4

| (2θ°) ± 0.2° | I(%) | d (A) |
|---|---|---|
| 6.737 | 73.7 | 13.1094 |
| 9.302 | 44.7 | 9.4995 |
| 9.494 | 36.1 | 9.3078 |
| 10.976 | 15.4 | 8.0543 |
| 11.442 | 8.7 | 7.7270 |
| 11.900 | 19.3 | 7.4310 |
| 12.801 | 15.7 | 6.9099 |
| 14.461 | 9.5 | 6.1201 |
| 14.800 | 18.3 | 5.9806 |
| 15.957 | 37.2 | 5.5494 |
| 17.240 | 100.0 | 5.1394 |
| 17.683 | 46.1 | 5.0116 |
| 18.520 | 46.3 | 4.7870 |
| 18.866 | 23.1 | 4.7000 |
| 19.103 | 7.4 | 4.6420 |
| 19.946 | 39.3 | 4.4478 |
| 20.502 | 13.2 | 4.3284 |
| 21.322 | 19.5 | 4.1637 |
| 21.896 | 10.1 | 4.0559 |
| 22.156 | 5.0 | 4.0089 |
| 22.824 | 22.5 | 3.8930 |
| 23.066 | 10.5 | 3.8527 |

In some preferred embodiments, the semi-dimethyl sulfoxide solvate crystalline form XIII of the compound 1 also has one or more of the following characteristics:

1) In the TGA diagram, there is a weight loss of 11.2±0.5% by weight before 80° C., and a weight loss of 8.0±0.5% by weight between 80° C. and 200° C., which is about half a dimethyl sulfoxide molecule and the decomposition temperature 266±2° C.; and/or

2) In the DSC diagram, there is a broad endothermic peak at 80° C.-160° C., which is caused by solvent removal. The melting point of the sample after solvent removal is 223±2° C.

In some preferred embodiments, the semi-dimethyl sulfoxide solvate crystalline form XIII of the compound 1 also has one or more of the following characteristics:

1) Basically the TGA diagram as shown in FIG. 40; and/or

2) Basically the DSC diagram as shown in FIG. 41.

14) The Compound 1 Semi-Methylcyclohexane Solvate Crystal Form XIV

In one embodiment, the form is the semi-methylcyclohexane solvate crystalline form XIV of the compound 1, which has characteristic peaks at the following positions in the XRPD diagram represented by 2θ angles: 4.134±0.2°, 7.102±0.2°, 7.982±0.2°, 14.301±0.2° and 16.701±0.2°.

In a preferred embodiment, the semi-methylcyclohexane solvate crystalline form XIV of the compound 1 has XRPD characteristic peaks at positions substantially as shown in Table 5 below and/or an XRPD pattern substantially as shown in FIG. 42.

TABLE 5

| (2θ°) ± 0.2° | I(%) | d (A) |
|---|---|---|
| 4.134 | 100.0 | 21.3544 |
| 4.601 | 13.8 | 19.1915 |
| 7.102 | 39.1 | 12.4370 |
| 7.981 | 35.4 | 11.0691 |
| 8.780 | 3.8 | 10.0635 |
| 9.203 | 9.9 | 9.6019 |
| 9.867 | 2.2 | 8.9568 |
| 10.779 | 0.6 | 8.2009 |
| 12.237 | 2.3 | 7.2272 |
| 12.740 | 10.6 | 6.9428 |
| 13.803 | 11.7 | 6.4104 |
| 14.301 | 27.8 | 6.1882 |
| 16.701 | 49.8 | 5.3041 |
| 17.100 | 13.5 | 5.1809 |
| 17.653 | 1.0 | 5.0200 |
| 18.886 | 7.6 | 4.6950 |
| 20.562 | 10.8 | 4.3159 |
| 21.442 | 3.7 | 4.1407 |
| 9.494 | 36.1 | 9.3078 |

In some preferred embodiments, the semi-methylcyclohexane solvate crystalline form XIV of the compound 1 also has one or more of the following characteristics:

1) In the TGA diagram, there is a weight loss of 8.62±0.20% by weight before 150° C., which is about half a methylcyclohexane molecule, and the decomposition temperature is 263±2° C.; and/or

2) In the DSC diagram, there is a broad endothermic peak at 45° C.-120° C., which is suspected to be caused by the removal of methylcyclohexane molecules.

In some preferred embodiments, the semi-methylcyclohexane solvent compound crystalline form XIV of the compound 1 further has one or more of the following characteristics:

1) Basically the TGA diagram as shown in FIG. 43; and/or

2) Basically the DSC diagram as shown in FIG. 44.

15) Semi-Tetrahydrofuran Solvate Crystalline Form XV of the Compound 1

In one embodiment, the form is the semi-tetrahydrofuran solvate crystalline form XV of the compound 1, which has characteristic peaks at the following positions in the XRPD diagram represented by 2θ angles: 7.961±0.2°, 8.402±0.2°, 12.739±0.2°, 13.242±0.2°, 17.164±0.2°, 17.625±0.2° and 19.540±0.2°.

In a preferred embodiment, the semi-tetrahydrofuran solvate crystalline form XV of the compound 1 has XRPD characteristic peaks at positions substantially as shown in Table 6 below and/or an XRPD pattern substantially as shown in FIG. 45.

TABLE 6

| (2θ°) ± 0.2° | I (%) | d (A) |
|---|---|---|
| 4.834 | 11.9 | 18.2636 |
| 7.961 | 87.2 | 11.0962 |
| 8.402 | 100.0 | 10.5151 |
| 11.527 | 1.4 | 7.6705 |
| 12.739 | 28.0 | 6.9430 |
| 13.242 | 38.9 | 6.6808 |
| 14.058 | 15.0 | 6.2947 |
| 14.622 | 30.7 | 6.0529 |
| 16.064 | 9.7 | 5.5129 |
| 16.680 | 1.6 | 5.3106 |
| 17.164 | 79.4 | 5.1618 |
| 17.625 | 36.0 | 5.0278 |
| 19.540 | 41.6 | 4.5392 |
| 20.476 | 7.5 | 4.3337 |
| 21.141 | 26.7 | 4.1990 |
| 21.984 | 35.7 | 4.0397 |
| 25.008 | 6.2 | 3.5577 |
| 28.964 | 5.5 | 3.0802 |
| 33.464 | 3.8 | 2.6755 |

In some preferred embodiments, the semi-tetrahydrofuran solvate crystalline form XV of the compound 1 further has one or more of the following characteristics:

1) In the TGA diagram, there is a weight loss of 6.8±0.2% by weight before 150° C., which is about half a tetrahydrofuran molecule, and the decomposition temperature is 265±2° C.; and/or

2) In the DSC diagram, there is a broad endothermic peak at 30° C.-150° C., which is suspected to be caused by the removal of tetrahydrofuran molecules, and the melting point is 197° C.±2° C.

In some preferred embodiments, the semi-tetrahydrofuran solvate crystalline form XV of the compound 1 further has one or more of the following characteristics:

1) Basically the TGA diagram as shown in FIG. 46; and/or

2) Basically the DSC diagram as shown in FIG. 47.

### 16) Amorphous Form XVI of Compound 1

In one embodiment, the form is the amorphous form XVI of the compound 1. In one embodiment, it has an XRPD pattern basically as shown in FIG. 48.

In a preferred embodiment, the amorphous form XVI of the compound 1 also has one or more of the following characteristics:

1) In the TGA diagram, there is a slow weight loss of 2.9±0.1% by weight before 150° C., and the decomposition temperature is 265±2° C.;

2) There is no melting peak in the DSC diagram; and/or

3) In the DVS diagram, the weight change from 0% RH to 80% RH is 2.5±0.5% (easy to absorb moisture).

In a preferred embodiment, the amorphous form XVI of the compound 1 also has one or more of the following characteristics:

1) Basically the TGA diagram as shown in FIG. 49;

2) Basically the DSC diagram as shown in FIG. 50; and/or

3) Basically the DVS diagram as shown in FIG. 51.

### 17) Crystal Form XVII of Compound 1

In one embodiment, the form is the crystalline form XVII of the compound 1, which has characteristic peaks at the following positions in the XRPD diagram represented by 2θ angles: 6.512±0.2°, 9.395±0.2°, 11.826±0.2°, 12.153±0.2°, 13.377±0.2°, 13.574±0.2°, 15.672±0.2° and 20.999±0.2°.

In a preferred embodiment, the crystalline form XVII of the compound 1 has XRPD characteristic peaks at positions substantially as shown in Table 7 below and/or an XRPD pattern substantially as shown in FIG. 52.

TABLE 7

| (2θ°) ± 0.2° | I (%) | d (A) |
|---|---|---|
| 5.783 | 15.2686 | 16.2 |
| 6.512 | 13.5612 | 28.9 |
| 8.361 | 10.5665 | 34.7 |
| 9.395 | 9.4057 | 51.8 |
| 10.539 | 8.3870 | 17.6 |
| 10.808 | 8.1789 | 10.2 |
| 11.826 | 7.4774 | 23.6 |
| 12.153 | 7.2764 | 36.5 |
| 12.868 | 6.8741 | 8.3 |
| 13.377 | 6.6133 | 97.4 |
| 13.574 | 6.5179 | 100.0 |
| 13.980 | 6.3294 | 14.2 |
| 14.742 | 6.0042 | 9.6 |
| 15.304 | 5.7848 | 19.5 |
| 15.672 | 5.6497 | 68.4 |
| 16.156 | 5.4815 | 34.1 |
| 16.668 | 5.3145 | 11.0 |
| 17.107 | 5.1789 | 41.0 |
| 17.383 | 5.0972 | 15.8 |
| 17.811 | 4.9757 | 27.4 |
| 18.119 | 4.8919 | 21.8 |
| 19.287 | 4.5983 | 12.8 |
| 19.892 | 4.4598 | 11.7 |
| 20.684 | 4.2906 | 34.0 |
| 20.999 | 4.2270 | 47.9 |
| 21.969 | 4.0425 | 10.5 |
| 22.627 | 3.9264 | 29.2 |
| 24.066 | 3.6948 | 12.6 |
| 24.556 | 3.6223 | 20.3 |
| 25.820 | 3.4476 | 10.6 |
| 26.808 | 3.3228 | 11.7 |
| 28.088 | 3.1742 | 13.3 |
| 29.703 | 3.0052 | 12.9 |

In a preferred embodiment, the crystalline form XVII of the compound 1 further has one or more of the following characteristics:

1) Basically the TGA diagram as shown in FIG. 53;

2) Basically the DSC diagram as shown in FIG. 54; and/or

3) Basically the DVS diagram as shown in FIG. 55.

### 18) The Crystalline Form XVIII of the Hydrochloride Salt of the Compound 1

In one embodiment, the form is the crystalline form XVIII of the hydrochloride salt of the compound 1, which has characteristic peaks at the following positions in the XRPD diagram represented by 2θ angles: 6.677±0.2°, 11.138±0.2°, 16.060±0.2°, 20.062±0.2°, 20.637±0.2°, and 21.559±0.2°.

In a preferred embodiment, the crystalline form XVIII of the compound 1 has XRPD characteristic peaks at positions substantially as shown in Table 8 below and/or an XRPD pattern substantially as shown in FIG. 56.

TABLE 8

| (2θ°) ± 0.2° | I (%) | d (A) |
|---|---|---|
| 6.677 | 100 | 13.2273 |
| 7.058 | 40.6 | 12.5142 |
| 11.138 | 65.4 | 7.9377 |
| 11.359 | 34.3 | 7.7836 |
| 12.001 | 33 | 7.3684 |
| 13.341 | 30.4 | 6.6312 |
| 14.419 | 41.7 | 6.1378 |
| 14.96 | 56 | 5.9171 |
| 16.06 | 85.7 | 5.5141 |
| 16.722 | 9.1 | 5.2973 |
| 17.38 | 41.1 | 5.0983 |
| 17.858 | 18 | 4.9629 |
| 18.139 | 25.5 | 4.8867 |
| 19.079 | 17.9 | 4.6479 |
| 20.062 | 63.2 | 4.4223 |
| 20.637 | 50.1 | 4.3003 |
| 21.203 | 15.2 | 4.1868 |
| 21.559 | 54.9 | 4.1184 |
| 22.001 | 12.3 | 4.0367 |
| 22.36 | 12.5 | 3.9727 |
| 22.82 | 24.5 | 3.8937 |
| 23.061 | 20.6 | 3.8536 |
| 23.823 | 13.4 | 3.732 |
| 24.637 | 8.9 | 3.6104 |
| 25.001 | 17.5 | 3.5587 |
| 25.5 | 30.7 | 3.4902 |
| 25.781 | 17.1 | 3.4528 |
| 26.222 | 10.2 | 3.3957 |
| 26.898 | 15.2 | 3.3119 |
| 27.861 | 26.9 | 3.1996 |
| 28.359 | 10.3 | 3.1445 |
| 28.76 | 14.1 | 3.1015 |
| 29.06 | 10.6 | 3.0702 |
| 29.921 | 17.3 | 2.9838 |
| 30.142 | 22.9 | 2.9624 |
| 30.561 | 15.2 | 2.9227 |
| 31.32 | 19.5 | 2.8537 |
| 32.001 | 16 | 2.7944 |
| 32.62 | 12.4 | 2.7429 |
| 33.88 | 8.1 | 2.6436 |
| 34.995 | 7.6 | 2.5619 |
| 35.563 | 8.9 | 2.5223 |
| 36.299 | 8.7 | 2.4728 |
| 36.839 | 9.3 | 2.4378 |
| 37.398 | 9.1 | 2.4026 |
| 37.856 | 8.4 | 2.3746 |
| 38.754 | 8.4 | 2.3216 |
| 39.244 | 7.2 | 2.2938 |

In a preferred embodiment, the crystalline form XVIII of the compound 1 further has one or more of the following characteristics:

1) Basically the TGA diagram as shown in FIG. 57; and/or

2) Basically the DSC diagram as shown in FIG. 58.

### 19) Amorphous Form of the Hydrobromide Salt of Formula 1 XIX

In one embodiment, the form is the amorphous form XIX of the hydrobromide salt of formula 1 compound. In one embodiment, it has an XRPD pattern substantially as shown in FIG. 59.

In a preferred embodiment, the hydrobromide salt amorphous form XIX of the compound 1 further has one or more of the following characteristics:

1) Basically the TGA diagram as shown in FIG. 60; and/or

2) Basically the DSC diagram as shown in FIG. 61.

### 20) The Hydrobromide Salt Crystalline Form XX of the Compound 1

In one embodiment, the form is the hydrobromide salt crystalline form XX of the compound 1, which has characteristic peaks at the following positions in the XRPD diagram represented by 2θ angles: 5.074±0.2°, 11.757±0.2°, 13.838±0.2°, 16.901±0.2°, 20.602±0.2°, and 25.440±0.2°.

In a preferred embodiment, the hydrobromide salt crystalline form XX of the compound 1 has XRPD characteristic peaks at positions substantially as shown in Table 9 below and/or an XRPD pattern substantially as shown in FIG. 62.

TABLE 9

| (2θ°) ± 0.2° | I (%) | d (A) |
|---|---|---|
| 5.074 | 38.1 | 17.4021 |
| 7.016 | 19.8 | 12.5885 |
| 8.89 | 20.9 | 9.9389 |
| 9.64 | 17.9 | 9.1675 |
| 10.119 | 38.2 | 8.7343 |
| 11.183 | 11.8 | 7.9057 |
| 11.757 | 46 | 7.5207 |
| 12.961 | 20.3 | 6.8246 |
| 13.838 | 44.9 | 6.3939 |
| 14.498 | 29.5 | 6.1045 |
| 15.335 | 13 | 5.7731 |
| 16.262 | 26.9 | 5.4461 |
| 16.901 | 100 | 5.2418 |
| 17.117 | 28.8 | 5.176 |
| 17.28 | 22.2 | 5.1274 |
| 17.878 | 22.7 | 4.9572 |
| 18.48 | 20.2 | 4.7971 |
| 18.802 | 39.8 | 4.7157 |
| 19.221 | 37.3 | 4.6139 |
| 19.618 | 27.9 | 4.5215 |
| 20.339 | 38.5 | 4.3627 |
| 20.602 | 59.6 | 4.3076 |
| 20.981 | 21.1 | 4.2306 |
| 21.542 | 17.9 | 4.1217 |
| 21.899 | 22.5 | 4.0553 |
| 22.444 | 16.9 | 3.9581 |
| 22.883 | 15.7 | 3.8831 |
| 23.515 | 31.1 | 3.7801 |
| 23.917 | 25.7 | 3.7175 |
| 24.298 | 51 | 3.6601 |
| 24.862 | 18.1 | 3.5783 |
| 25.44 | 42.6 | 3.4984 |
| 26.042 | 26.2 | 3.4188 |
| 26.879 | 26.7 | 3.3142 |
| 27.318 | 30.1 | 3.2619 |
| 28.095 | 29.6 | 3.1734 |
| 28.477 | 17.8 | 3.1317 |
| 29.161 | 21.5 | 3.0598 |
| 29.48 | 20.8 | 3.0275 |
| 30.181 | 18.4 | 2.9587 |
| 30.676 | 16.8 | 2.912 |
| 31.041 | 17 | 2.8786 |
| 31.337 | 16.8 | 2.8521 |
| 31.541 | 18.8 | 2.8342 |
| 31.896 | 18.3 | 2.8034 |
| 32.158 | 21.3 | 2.7812 |
| 32.962 | 17.3 | 2.7152 |
| 33.345 | 16.6 | 2.6849 |
| 34.005 | 16.4 | 2.6342 |
| 35.396 | 20.3 | 2.5338 |
| 36.236 | 15.2 | 2.477 |
| 36.481 | 20.3 | 2.4609 |
| 37.056 | 16 | 2.424 |
| 37.597 | 14.5 | 2.3904 |
| 38.158 | 16.1 | 2.3565 |
| 38.517 | 15.2 | 2.3354 |
| 39.279 | 14.9 | 2.2918 |

In a preferred embodiment, the hydrobromide salt crystalline form XX of the compound 1 also has one or more of the following characteristics:

1) Basically the TGA diagram as shown in FIG. 63; and/or

2) Basically the DSC diagram as shown in FIG. 64.

### 21) The Hydrobromide Salt Crystalline Form XXI of Compound 1

In one embodiment, the form is the hydrobromide salt crystalline form XXI of the compound 1, which has characteristic peaks at the following positions in the XRPD diagram represented by 2θ angles: 8.141±0.2°, 8.695±0.2°, 12.157±0.2°, 12.805±0.2°, 13.860±0.2°, and 17.263±0.2°.

In a preferred embodiment, the hydrobromide salt crystalline form XXI of the compound 1 has XRPD characteristic peaks at positions substantially as shown in Table 10 below and/or an XRPD pattern substantially as shown in FIG. 65.

TABLE 10

| (2θ°) ± 0.2° | I (%) | d (A) |
|---|---|---|
| 8.141 | 100 | 10.8509 |
| 8.695 | 75.8 | 10.1614 |
| 12.157 | 70.6 | 7.2744 |
| 12.805 | 42.9 | 6.9077 |
| 13.119 | 30.6 | 6.7432 |
| 13.86 | 41.5 | 6.3841 |
| 15.421 | 28 | 5.741 |
| 17.263 | 46.4 | 5.1325 |
| 18.28 | 33.8 | 4.8492 |
| 18.663 | 35.1 | 4.7506 |
| 20.905 | 28.3 | 4.2459 |
| 21.499 | 30 | 4.1298 |
| 21.883 | 27.8 | 4.0583 |
| 22.535 | 31.3 | 3.9422 |
| 25.055 | 26.4 | 3.5511 |
| 26.315 | 26.5 | 3.3839 |
| 26.58 | 28.7 | 3.3507 |
| 28.474 | 27 | 3.1321 |
| 31.14 | 25.5 | 2.8697 |
| 39.356 | 21.1 | 2.2875 |

In a preferred embodiment, the hydrobromide salt crystalline form XXI of the compound 1 further has one or more of the following characteristics:

1) Basically the TGA diagram shown in FIG. 66; and/or

2) Basically the DSC diagram shown in FIG. 67.

22) The hydrobromide salt crystalline form XXII of compound 1

In one embodiment, the form is the hydrobromide salt crystalline form XXII of the compound 1, which has characteristic peaks at the following positions in the XRPD diagram represented by 2θ angles: 6.557±0.2°, 6.900±0.2°, 15.920±0.2°, 17.140±0.2°, 17.781±0.2°, and 19.860±0.2°.

In a preferred embodiment, the hydrobromide salt crystalline form XXII of the compound 1 has XRPD characteristic peaks at positions substantially as shown in Table 11 below and/or an XRPD pattern substantially as shown in FIG. 68.

TABLE 11

| (2θ°) ± 0.2° | I (%) | d (A) |
|---|---|---|
| 6.557 | 100 | 13.4686 |
| 6.9 | 47 | 12.7994 |
| 11.119 | 35.3 | 7.9507 |
| 11.855 | 17.4 | 7.4588 |
| 13.201 | 24.4 | 6.701 |
| 14.301 | 23.5 | 6.1884 |
| 14.781 | 38 | 5.9881 |
| 15.92 | 54.4 | 5.5622 |
| 16.481 | 19.1 | 5.3744 |
| 17.14 | 72.3 | 5.1689 |
| 17.781 | 40.6 | 4.9841 |
| 19.06 | 22.9 | 4.6524 |
| 19.86 | 56.3 | 4.4668 |
| 20.279 | 36.6 | 4.3755 |
| 21.46 | 34.7 | 4.1374 |
| 22.64 | 28.3 | 3.9242 |
| 23.6 | 29.4 | 3.7668 |
| 24.323 | 19.6 | 3.6563 |
| 24.942 | 27.2 | 3.5671 |
| 25.18 | 37.1 | 3.5338 |
| 25.741 | 22.1 | 3.4581 |
| 26.42 | 22 | 3.3707 |
| 27.56 | 25 | 3.2338 |
| 27.937 | 23.7 | 3.191 |
| 28.44 | 27.3 | 3.1358 |
| 28.857 | 19.8 | 3.0914 |
| 29.76 | 27 | 2.9996 |
| 30.239 | 22 | 2.9531 |
| 30.92 | 25.1 | 2.8896 |
| 31.4 | 24.4 | 2.8466 |
| 32.06 | 18.5 | 2.7895 |
| 33.297 | 14.6 | 2.6886 |
| 34.481 | 14.8 | 2.599 |
| 35.181 | 18.6 | 2.5488 |
| 36.314 | 16.8 | 2.4718 |
| 37.02 | 19.2 | 2.4263 |
| 38.023 | 14.6 | 2.3646 |
| 38.682 | 14.5 | 2.3258 |
| 39.299 | 15.8 | 2.2907 |

In a preferred embodiment, the hydrobromide salt crystalline form XXII of the compound 1 further has one or more of the following characteristics:

1) Basically the TGA diagram shown in FIG. 69; and/or

2) Basically the DSC diagram shown in FIG. 70.

23) The Crystalline Form XXIII of the Mesylate Salt of the Compound 1

In one embodiment, the form is the crystalline form XXIII of the mesylate salt of the compound 1, which has characteristic peaks at the following positions in the XRPD diagram represented by 2θ angles: 5.203±0.2°, 9.640±0.2°, 13.970±0.2°, 16.731±0.2° and 19.716±0.2°.

In a preferred embodiment, the mesylate salt crystalline form XXIII of the compound 1 has XRPD characteristic peaks at positions substantially as shown in Table 12 below and/or an XRPD pattern substantially as shown in FIG. 71.

TABLE 12

| (2θ°) ± 0.2° | d (A) | I (%) |
|---|---|---|
| 5.203 | 16.9708 | 55.4 |
| 5.391 | 16.3781 | 14.5 |
| 6.915 | 12.7723 | 8.6 |
| 8.664 | 10.1976 | 2.9 |
| 9.152 | 9.6548 | 11.2 |
| 9.64 | 9.1673 | 34.4 |
| 10.186 | 8.6767 | 3.2 |
| 10.604 | 8.3357 | 9.6 |
| 11.372 | 7.7743 | 6.6 |
| 11.782 | 7.5051 | 17.9 |
| 12.154 | 7.2761 | 5.4 |
| 13.012 | 6.7984 | 14.3 |
| 13.3 | 6.6517 | 27.9 |
| 13.97 | 6.3342 | 56.8 |
| 14.26 | 6.206 | 53 |
| 15.234 | 5.8113 | 21.7 |
| 15.564 | 5.6888 | 23.7 |
| 15.854 | 5.5853 | 26.7 |
| 16.236 | 5.4548 | 4.9 |
| 16.731 | 5.2945 | 100 |
| 17.224 | 5.1441 | 14.3 |
| 17.555 | 5.0478 | 20.3 |
| 17.986 | 4.9279 | 30 |
| 18.231 | 4.862 | 32.6 |
| 18.567 | 4.7748 | 5 |
| 19.325 | 4.5892 | 55.8 |
| 19.716 | 4.4992 | 66.6 |
| 20.298 | 4.3714 | 28.1 |
| 21.188 | 4.1897 | 19 |
| 21.444 | 4.1403 | 14 |
| 21.839 | 4.0664 | 29.4 |
| 22.575 | 3.9354 | 5.7 |
| 23.223 | 3.827 | 14.6 |
| 23.762 | 3.7414 | 15.2 |
| 24.135 | 3.6845 | 5.6 |
| 24.469 | 3.6349 | 14.7 |
| 24.799 | 3.5872 | 18.1 |
| 25.012 | 3.5572 | 10.7 |
| 25.255 | 3.5235 | 8.6 |
| 26.044 | 3.4185 | 27.2 |
| 26.629 | 3.3448 | 8 |
| 27.238 | 3.2713 | 8.9 |
| 27.66 | 3.2223 | 7.8 |
| 27.916 | 3.1934 | 12.4 |
| 28.19 | 3.163 | 16.2 |
| 28.837 | 3.0935 | 11.4 |
| 29.356 | 3.0399 | 9.1 |
| 30.155 | 2.9612 | 6.9 |
| 30.483 | 2.9301 | 17.2 |
| 30.89 | 2.8924 | 3.8 |
| 31.344 | 2.8516 | 11.3 |
| 31.846 | 2.8077 | 5.6 |
| 32.165 | 2.7806 | 4.9 |
| 32.827 | 2.726 | 6.1 |
| 33.789 | 2.6506 | 4.4 |
| 34.441 | 2.6019 | 5.4 |
| 35.226 | 2.5457 | 3.8 |
| 35.971 | 2.4946 | 3.8 |
| 36.272 | 2.4746 | 5 |
| 37.227 | 2.4133 | 3.4 |
| 37.455 | 2.3991 | 4.2 |
| 37.88 | 2.3732 | 4.5 |
| 38.281 | 2.3492 | 6.9 |
| 39.08 | 2.303 | 5.6 |
| 39.45 | 2.2823 | 3.9 |

In a preferred embodiment, the crystalline form XXIII of the mesylate salt of the compound 1 further has one or more of the following characteristics:

1) Basically the TGA diagram as shown in FIG. 72; and/or

2) Basically the DSC diagram as shown in FIG. 73.

### 24) The Crystalline Form XXIV of the Mesylate Salt of the Compound 1

In one embodiment, the form is the crystalline form XXIV of the mesylate salt of the compound 1, which has characteristic peaks at the following positions in the XRPD diagram represented by 2θ angles: 12.235±0.2°, 17.980±0.2°, 18.584±0.2° and 20.511±0.2°.

In a preferred embodiment, the mesylate salt crystalline form XXIV of the compound 1 has XRPD characteristic peaks at positions substantially as shown in Table 13 below and/or an XRPD pattern substantially as shown in FIG. 74.

TABLE 13

| (2θ°) ± 0.2° | d (A) | I (%) |
|---|---|---|
| 6.192 | 14.261 | 13.3 |
| 7.614 | 11.6012 | 17.8 |
| 9.369 | 9.4321 | 18 |
| 10.288 | 8.5916 | 9.5 |
| 11.277 | 7.8399 | 40.5 |
| 11.592 | 7.6277 | 47.9 |
| 12.235 | 7.2283 | 57 |
| 13.559 | 6.525 | 19.1 |
| 14.08 | 6.2847 | 4.9 |
| 15.077 | 5.8714 | 4.1 |
| 15.773 | 5.6137 | 9.6 |
| 16.5 | 5.368 | 16.4 |
| 16.811 | 5.2694 | 10.1 |
| 17.98 | 4.9295 | 45.4 |
| 18.31 | 4.8413 | 70.1 |
| 18.584 | 4.7706 | 78.7 |
| 19.172 | 4.6256 | 13.8 |
| 19.556 | 4.5356 | 19.9 |
| 19.83 | 4.4735 | 19 |
| 20.511 | 4.3266 | 100 |
| 22.616 | 3.9283 | 25.3 |
| 23.102 | 3.8467 | 40.4 |
| 23.864 | 3.7257 | 37.8 |
| 24.655 | 3.6078 | 10.7 |
| 25.105 | 3.5442 | 10.4 |
| 25.777 | 3.4533 | 15.2 |
| 26.104 | 3.4108 | 5.2 |
| 26.978 | 3.3023 | 6.7 |
| 27.634 | 3.2254 | 4.5 |
| 28.341 | 3.1464 | 23.1 |
| 28.73 | 3.1048 | 11.9 |
| 30.043 | 2.9719 | 5.8 |
| 30.973 | 2.8848 | 10.1 |
| 32.859 | 2.7234 | 7.1 |
| 33.97 | 2.6368 | 11.7 |
| 34.715 | 2.5819 | 5.9 |
| 38.872 | 2.3148 | 5 |

In a preferred embodiment, the crystalline form XXIV of the mesylate salt of the compound 1 further has one or more of the following characteristics:

1) Basically the TGA diagram as shown in FIG. 75; and/or

2) Basically the DSC diagram as shown in FIG. 76.

### 25) The Crystalline Form XXV of the Sulfate Salt of the Compound 1

In one embodiment, the form is the crystalline form XXV of the sulfate salt of the compound 1, which has characteristic peaks at the following positions in the XRPD diagram represented by 2θ angles: 4.054±0.2°, 11.785±0.2°, 13.286±0.2° and 15.680±0.2°.

In a preferred embodiment, the sulfate salt crystalline form XXV of the compound 1 has characteristic XRPD peaks at positions substantially as shown in Table 14 below and/or an XRPD pattern substantially as shown in FIG. 77.

TABLE 14

| (2θ°) ± 0.2 | d (A) | I (%) |
|---|---|---|
| 4.054 | 21.7787 | 65.7 |
| 6.332 | 13.9478 | 5.5 |
| 6.624 | 13.3323 | 18.6 |
| 7.904 | 11.1768 | 4.8 |
| 8.769 | 10.0755 | 23.8 |
| 10.109 | 8.743 | 19 |
| 10.68 | 8.2769 | 16 |
| 11.161 | 7.9212 | 10.5 |
| 11.785 | 7.5031 | 57.9 |
| 12.581 | 7.0302 | 33.3 |
| 13.286 | 6.6583 | 49.9 |
| 14.145 | 6.256 | 17.5 |
| 14.531 | 6.0906 | 22.3 |
| 15.68 | 5.6468 | 100 |
| 16.322 | 5.4261 | 20.8 |
| 16.947 | 5.2276 | 36.2 |
| 18.153 | 4.8829 | 18.7 |
| 18.507 | 4.7902 | 38.2 |
| 18.739 | 4.7313 | 38.6 |
| 19.031 | 4.6594 | 6.7 |
| 19.79 | 4.4825 | 48.3 |
| 20.302 | 4.3707 | 13.9 |
| 20.573 | 4.3136 | 16.9 |
| 21.036 | 4.2196 | 15.8 |
| 21.344 | 4.1595 | 8 |
| 22.053 | 4.0272 | 15.6 |
| 23.045 | 3.8562 | 10.1 |
| 23.689 | 3.7527 | 7.6 |
| 24.424 | 3.6415 | 11.8 |
| 24.814 | 3.5851 | 8.8 |
| 25.654 | 3.4696 | 11 |
| 26.45 | 3.367 | 7.3 |
| 26.72 | 3.3335 | 8.7 |
| 26.867 | 3.3156 | 10.6 |
| 27.465 | 3.2448 | 15.7 |
| 27.711 | 3.2165 | 13.2 |
| 28.516 | 3.1275 | 6.6 |
| 29.934 | 2.9825 | 5.2 |
| 31.265 | 2.8586 | 6.9 |
| 32.573 | 2.7467 | 6.2 |

In a preferred embodiment, the crystalline form XXV of the compound 1 sulfate salt further has one or more of the following characteristics:

1) Basically the TGA diagram as shown in FIG. 78; and/or

2) Basically the DSC diagram as shown in FIG. 79.

### 26) The Crystalline Form XXVI of the Compound 1 Sulfate Salt

In one embodiment, the form is the crystalline form XXVI of the compound 1 sulfate salt, which has characteristic peaks at the following positions in the XRPD diagram represented by 2θ angles: 7.266±0.2°, 9.275±0.2°, 10.713±0.2°, 14.219±0.2° and 18.583±0.2°.

In a preferred embodiment, the crystalline form XXVI of the compound 1 sulfate salt has XRPD characteristic peaks at positions substantially as shown in Table 15 below and/or an XRPD pattern substantially as shown in FIG. 80.

TABLE 15

| (2θ°) ± 0.2 | d (A) | I (%) |
|---|---|---|
| 7.266 | 12.1561 | 100 |
| 9.275 | 9.5274 | 42.6 |
| 10.713 | 8.2509 | 77.8 |
| 12.231 | 7.2305 | 48.3 |
| 12.701 | 6.9641 | 37.8 |
| 13.033 | 6.7872 | 20.4 |
| 13.62 | 6.4959 | 30 |
| 14.219 | 6.2236 | 51.7 |
| 16.146 | 5.485 | 36.1 |
| 17.106 | 5.1794 | 39.1 |
| 17.531 | 5.0547 | 40.4 |
| 18.096 | 4.898 | 53 |
| 18.583 | 4.7708 | 70 |
| 19.831 | 4.4734 | 30.9 |
| 21.329 | 4.1623 | 34.3 |
| 21.617 | 4.1075 | 22.2 |
| 25.268 | 3.5218 | 19.6 |
| 25.788 | 3.4519 | 18.3 |
| 30.489 | 2.9295 | 17.8 |
| 35.256 | 2.5435 | 19.1 |

In a preferred embodiment, the crystalline form XXVI of the compound 1 sulfate salt further has one or more of the following characteristics:

1) Basically the TGA diagram as shown in FIG. 81; and/or

2) Basically the DSC diagram as shown in FIG. 82.

Second, the present invention provides a method for preparing the amorphous or crystalline form of the compound 1 or its salt or solvate.

In one embodiment, the present invention provides a method for preparing the amorphous or crystalline form of the salt of the compound 1, which comprises the following steps: reacting the compound 1 with an acid or base in an organic solvent, and then preparing the corresponding salt shaped form or crystalline form. The preparation method of the crystalline or amorphous form of the salt of the compound 1 can be a method well known in the art, such as suspension stirring, normal temperature or stirring, heating and cooling for crystallization, solvent volatilization or anti-solvent addition.

In the preparation method, the compound 1 can be obtained through various channels, such as commercial purchase or laboratory synthesis. The acid may be a pharmaceutically acceptable acid or an acid commonly used in the art, and may be an inorganic acid or an organic acid. The inorganic acid is preferably hydrochloric acid, hydrobromic acid, sulfuric acid or phosphoric acid. The organic acid is preferably methanesulfonic acid, p-toluenesulfonic acid, maleic acid, L-tartaric acid, fumaric acid, citric acid, malic acid or succinic acid, more preferably hydrobromic acid, L-tartaric acid, fumaric acid, and maleic acid. Hydrobromic acid and maleic acid are further selected. The molar ratio of the compound 1 to the acid is 1:(1-1.5), preferably 1:(1-1.2).

In the preparation method, the organic solvent can be an organic solvent commonly used in laboratories, such as: alkane solvents, alcohol solvents, ketone solvents, ester solvents, aromatic hydrocarbon solvents, halogenated hydrocarbon solvents, nitriles One or more of solvents, ether solvents, aliphatic hydrocarbon solvents, polar aprotic solvents such as DMF and DMSO, preferably C1-C6 alcohols, ketone solvents, ester solvents, more preferably methanol, ethanol, Isopropanol, acetone, 2-butanone, ethyl acetate, isopropyl acetate. The mass-volume ratio of the compound 1 to the organic solvent is 100 mg: (0.1-1 mL), preferably 100 mg: (0.4-1 mL), more preferably 100 mg: 0.6 mL, 100 mg: 0.8 mL.

In the preparation method, the reaction temperature may be room temperature to solvent reflux temperature.

In the preparation method, the crystallization time is not particularly limited, as long as the crystals can be precipitated, and the reaction time can be 1 hour to 36 hours.

In one embodiment, the present invention also provides a method for preparing the amorphous or crystalline form of the salt of the compound 1, which preferably comprises the following steps: mixing the compound 1 with an organic solvent, and then adding acid and organic solvent, and mixing the liquid, stir well and filter. The mixing before adding the acid is preferably carried out under stirring. After the filtration is completed, drying is preferably included. The drying is preferably vacuum drying, and the drying temperature is preferably 40-60° C., for example, 50° C.

In one embodiment, the present invention also provides a method for preparing the amorphous or crystalline form of the salt of the compound 1, which comprises the following steps: reacting the compound 1 with a base in an organic solvent.

In the preparation method, the organic solvent may be an organic solvent commonly used in laboratories, such as: alkane solvents, alcohol solvents, ketone solvents, preferably alcohol solvents, more preferably methanol, ethanol, isopropanol, wherein The mass-volume ratio of the compound 1 to the organic solvent is 100 mg: (0.1-1 mL), preferably 100 mg: (0.4-1 mL), more preferably 100 mg: 0.6 mL, 100 mg: 0.8 mL.

In the preparation method, the base is an alkali metal hydroxide commonly used in the art, such as: LiOH, NaOH, KOH, and the molar ratio of the compound 1 to the base is 1:(1-1.5), preferably 1:(1-1.2).

In one embodiment, the present invention also provides a method for preparing the amorphous or crystalline form of the solvate of the compound 1, which comprises the following steps: contacting or reacting the compound 1 with a solvent, and then preparing the corresponding amorphous or crystalline form. The preparation method of the amorphous or crystalline form of the solvate of the compound 1 can be a method well known in the art, such as suspension stirring, normal temperature or stirring, heating and cooling crystallization, solvent volatilization or mixed solvent crystallization.

In the preparation method, the solvent is preferably one or more of water, isopropyl ether, trifluoroethanol, acetonitrile, dimethyl sulfoxide, tetrahydrofuran, ethyl acetate, toluene, and methylcyclohexane, Wherein the mass-volume ratio of the compound 1 to the solvent is 100 mg: (1-15 mL), preferably 100 mg: (2-12 mL).

In the preparation method, the temperature of the crystallization may be a temperature conventional in the art, such as 20-50° C.

In the preparation method, the crystallization time is not particularly limited, as long as the crystals can be precipitated, for example, 1-48 hours.

In one embodiment, the present invention also provides a method for preparing the amorphous or crystalline form of the compound 1, which comprises the following steps: contacting or reacting the compound 1 with a solvent, and then preparing the corresponding amorphous form or crystalline form. The preparation method of the amorphous form or the crystalline form of the compound 1 may be a method well known in the art, such as suspension stirring, normal temperature or stirring, heating and cooling for crystallization, solvent volatilization method or antisolvent addition method.

In the preparation method, the solvent may be water or organic solvents commonly used in laboratories in the field, such as: one or more of alkane solvents, alcohol solvents, ketone solvents, ester solvents, aromatic hydrocarbon solvents, halogenated hydrocarbons solvents, nitrile solvents, ether solvents, aliphatic hydrocarbon solvents, acetonitrile, DMF and DMSO, preferably alkane solvents, alcohol solvents, ketone solvents, ester solvents, halogenated hydrocarbon solvents, Ether solvents, acetonitrile, nitromethane, aromatic hydrocarbon solvents, more preferably one or more of n-heptane, methanol, ethanol, n-propanol, isopropanol, n-butanol, trifluoroethanol, acetone, 2-butanone, ethyl acetate, isopropyl acetate, isopropyl ether, tetrahydrofuran, 1,4-dioxane, dichloromethane, chloroform, acetonitrile, nitromethane, toluene, DMF and DMSO. The mass-volume ratio of the compound 1 to the organic solvent is 100 mg: (0.1-3 mL).

In the preparation method, the temperature of the crystallization may be a temperature conventional in the art, such as 20-50° C.

In the preparation method, the crystallization time is not particularly limited, as long as the crystals can be precipitated, for example, 1-48 h.

The solvent volatilization method of the present invention is to volatilize the clear sample solution at different temperatures until the solvent is evaporated to dryness.

The suspension stirring in the present invention is to stir the supersaturated solution of the sample (with insoluble solids) in different solvents for a period of time.

The heating and cooling crystallization in the present invention is to dissolve the sample in an appropriate solvent under high temperature conditions, and after filtering, the filtrate is stirred and precipitated in a room temperature or low temperature environment.

The mixed solvent crystallization method of the present invention is to take a sample and dissolve it in a suitable solvent, add another or more solvents, and precipitate a solid system for a short time after stirring and filtering.

Third, the present invention provides a pharmaceutical composition comprising the above-mentioned amorphous or crystalline form of compound 1 or its salt, solvate and pharmaceutically acceptable excipients.

The amorphous or crystalline form of the compound 1 or its salt or solvate may be a therapeutically effective amount. The pharmaceutically acceptable excipients may be well-known excipients in the art. In the case of solid preparations, they include, but are not limited to: diluents, binders, disintegrants, lubricants, glidants, release rate control agents, plasticizers, preservatives, antioxidants, etc.

The pharmaceutical composition can be selected in a dosage form suitable for human consumption, such as: tablets, capsules, granules, powders, or pills, etc., preferably tablets, capsules, granules, disintegrating tablets, sustained release or controlled release tablets, etc.

The pharmaceutical composition of the present invention can be prepared by various methods well-known in the art, which can combine a therapeutically effective amount of one or more of the compound 1 or its salt or solvate in the amorphous or crystalline form with one or more pharmaceutically acceptable excipients to prepare dosage forms suitable for human consumption, such as tablets, capsules, and granules.

A “therapeutically effective amount” is the amount of a compound in the form of the present invention that, when administered to a patient in need, is sufficient to achieve treatment of a disease state, condition, or disorder for which the compound has utility. Such a quantity would be sufficient to elicit a biological or medical response in the tissue system or patient sought by researchers or clinicians.

Fourth, the present invention provides the use of amorphous or crystalline form of the compound 1 or its salt, solvate, or the above-mentioned pharmaceutical composition in the preparation of drugs for the prevention and/or treatment of hyperproliferative diseases.

In one embodiment, the drug is preferably used to prevent and/or treat cancer, including but not limited to adrenal cortical cancer, advanced cancer, anal cancer, aplastic anemia, cholangiocarcinoma, bladder cancer, bone cancer, Bone metastasis, adult brain/CNS tumor, children brain/CNS tumor, breast cancer, male breast cancer, childhood cancer, unknown primary cancer, giant lymph node hyperplasia (Castleman disease), cervical cancer, colon/rectal cancer, uterus Endometrial cancer, esophageal cancer, Ewing family of tumors, eye cancer, gallbladder cancer, gastrointestinal carcinoid tumor, gastrointestinal stromal tumor (GIST), gestational trophoblastic disease, Hodgkin's Hodgkin disease, Kaposisarcoma, kidney cancer, laryngeal and hypopharyngeal cancer, adult acute lymphoblastic leukemia (ALL), acute myelogenous leukemia (AML), chronic lymphocytic leukemia (CLL), Chronic myelogenous leukemia (CML), chronic myelogenous leukemia (CMML), childhood leukemia, liver cancer, non-small cell lung cancer, small cell lung cancer, lung carcinoid tumor, skin lymphoma, malignant mesothelioma, multiple bone marrow Tumor, myelodysplastic syndrome, nasal cavity and paranasal sinus cancer, nasopharyngeal carcinoma, neuroblastoma, non-Hodgkin's lymphoma, childhood non-Hodgkin's lymphoma, oral and oropharyngeal cancer, osteosarcoma, ovarian cancer, Pancreatic cancer, penile cancer, pituitary tumor, prostate cancer, retinoblastoma, rhabdomyosarcoma, salivary gland cancer, sarcoma-adult soft tissue cancer, basal skin cancer and squamous cell skin cancer, skin cancer-melanoma, small intestine cancer, gastric cancer, Testicular cancer, thymic cancer, thyroid cancer, uterine sarcoma, vaginal cancer, vulvar cancer, Waldenstrom macroglobulinemia or Wilms Tumor.

The amorphous or crystalline form of formula 1 compound or its salt and solvate of the invention has the following advantages:

1. The invention discovers for the first time a variety of amorphous form or crystalline form of formula 1 compound or its salt and solvate that have not been reported, and the said form can be used as an important basis for subsequent drug development, preparation development and production.

2. Through a large number of experiments and screens, the invention selects forms V, VI, XI and XVI as candidate objects. The forms V, VI, XI and XVI have good physical stability, are easy to store, can avoid the risk of crystallization during drug development or production, avoid changes in bioavailability and efficacy, and can then be developed into dosage forms suitable for clinical use and commercial production. Moreover, its preparation method is simple, reproducible, and has high development value.

### BRIEF DESCRIPTION OF THE DRAWINGS

FIG. 1 is the XRD pattern of the amorphous form I of the sulfate salt of the compound 1.

FIG. 2 is a TGA diagram of the amorphous form I of the sulfate salt of the compound 1.

FIG. 3 is a DSC diagram of the sulfate salt amorphous form I of the compound 1.

FIG. 4 is a DVS diagram of the amorphous form I of the sulfate salt of the compound 1.

FIG. 5 is an Isotherm adsorption curve of the compound 1 sulfate amorphous form I.

FIG. 6 is the XRD pattern of the amorphous form II of the hydrochloride salt of the compound 1.

FIG. 7 is the XPRD pattern of the crystalline form III of the hydrochloride salt of the compound 1.

FIG. 8 is a TGA diagram of the crystalline form III of the hydrochloride salt of the compound 1.

FIG. 9 is a DSC diagram of the crystalline form III of the hydrochloride salt of the compound 1.

FIG. 10 is the XPRD pattern of the crystalline form IV of the hydrochloride salt of the compound 1.

FIG. 11 is the XPRD pattern of the crystalline form V of the maleate salt of the compound 1

FIG. 12 is a TGA diagram of the crystalline form V of the maleate salt of the compound 1.

FIG. 13 is a DSC diagram of the crystalline form V of the maleate salt of the compound 1.

FIG. 14 is a DVS diagram of the crystalline form V of the maleate salt of the compound 1.

FIG. 15 is the XPRD pattern of the crystalline form VI of the hydrobromide salt of the compound 1.

FIG. 16 is a TGA diagram of the crystalline form VI of the hydrobromide salt of the compound 1.

FIG. 17 is a DSC diagram of the crystalline form VI of the hydrobromide salt of the compound 1.

FIG. 18 is a DVS diagram of the crystalline form VI of the hydrobromide salt of the compound 1.

FIG. 19 is an XRD pattern of the amorphous form VII of the mesylate salt of the compound 1.

FIG. 20 is the XRD pattern of the sodium salt amorphous form VIII of the compound 1.

FIG. 21 is a TGA diagram of the sodium salt amorphous form VIII of the compound 1.

FIG. 22 is a DSC diagram of the sodium salt amorphous form VIII of the compound 1.

FIG. 23 is a DVS diagram of the sodium salt amorphous form VIII of the compound 1.

FIG. 24 is an XRD pattern of the amorphous form IX of the potassium salt of the compound 1

FIG. 25 is a TGA diagram of the amorphous form IX of the potassium salt of the compound 1

FIG. 26 is a DSC diagram of the potassium salt amorphous form IX of the compound 1.

FIG. 27 is a DVS diagram of the potassium salt amorphous form IX of the compound 1.

FIG. 28 is an XPRD pattern of the crystalline form X of the compound 1.

FIG. 29 is a TGA diagram of the crystalline form X of the compound 1.

FIG. 30 is a DSC diagram of the crystalline form X of the compound 1.

FIG. 31 is a DVS diagram of the crystalline form X of the compound 1.

FIG. 32 is the XPRD pattern of the crystalline form XI of the compound 1 monohydrate.

FIG. 33 is a TGA diagram of the crystalline form XI of the compound 1 monohydrate.

FIG. 34 is a DSC diagram of the crystalline form XI of the compound 1 monohydrate.

FIG. 35 is a DVS diagram of the crystalline form XI of the compound 1 monohydrate.

FIG. 36 is the XPRD pattern of the crystalline form XII of the di-trifluoroethanol solvate of the compound 1.

FIG. 37 is a TGA diagram of the crystalline form XII of the di-trifluoroethanol solvate of the compound 1.

FIG. 38 is a DSC diagram of the crystalline form XII of the di-trifluoroethanol solvate compound 1.

FIG. 39 is the XPRD pattern of the crystalline form XIII of the semi-dimethyl sulfoxide solvent compound 1.

FIG. 40 is a TGA diagram of the crystalline form XIII of the semi-dimethyl sulfoxide solvent compound 1.

FIG. 41 is a DSC diagram of the crystalline form XIII of the semi-dimethyl sulfoxide solvent compound 1.

FIG. 42 is the XPRD pattern of the crystalline form XIV of the semi-methylcyclohexane solvent compound 1.

FIG. 43 is a TGA diagram of the crystalline form XIV of the semi-methylcyclohexane solvent compound 1.

FIG. 44 is a DSC diagram of the crystalline form XIV of the semi-methylcyclohexane solvent compound 1.

FIG. 45 is the XPRD pattern of the crystalline form XV of the semi-tetrahydrofuran solvent compound 1.

FIG. 46 is a TGA diagram of the crystalline form XV of the semi-tetrahydrofuran solvent compound 1.

FIG. 47 is a DSC diagram of the crystalline form XV of the semi-tetrahydrofuran solvent compound 1.

FIG. 48 is an XRD pattern of the amorphous form XVI of the compound 1.

FIG. 49 is a TGA diagram of the amorphous form XVI of the compound 1.

FIG. 50 is a DSC diagram of the amorphous form XVI of the compound 1.

FIG. 51 is a DVS diagram of the amorphous form XVI of the compound 1.

FIG. 52 is an XRD pattern of the crystalline form XVII of the compound 1.

FIG. 53 is a TGA diagram of the crystalline form XVII of the compound 1.

FIG. 54 is a DSC diagram of the crystalline form XVII of the compound 1.

FIG. 55 is a DVS diagram of the crystalline form XVII of the compound 1.

FIG. 56 is an XRD pattern of the crystalline form XVIII of the hydrochloride salt of the compound 1.

FIG. 57 is a TGA diagram of the crystalline form XVIII of the hydrochloride salt of the compound 1.

FIG. 58 is a DSC diagram of the crystalline form XVIII of the hydrochloride salt of the compound 1.

FIG. 59 is the XRD pattern of the amorphous form XIX of the hydrobromide salt of the compound 1.

FIG. 60 is a TGA diagram of the amorphous form of the hydrobromide salt of formula 1 XIX.

FIG. 61 is a DSC diagram of the amorphous form XIX of the hydrobromide salt of the compound 1.

FIG. 62 is the XRD pattern of the crystalline form XX of the hydrobromide salt of the compound 1.

FIG. 63 is a TGA diagram of the crystalline form XX of the hydrobromide salt of the compound 1.

FIG. 64 is a DSC diagram of the crystalline form XX of the hydrobromide salt of the compound 1.

FIG. 65 is an XRD pattern of the crystalline form XXI of the hydrobromide salt of the compound 1.

FIG. 66 is a TGA diagram of the crystalline form XXI of the hydrobromide salt of the compound 1.

FIG. 67 is a DSC diagram of the crystalline form XXI of the hydrobromide salt of the compound 1.

FIG. 68 is the XRD pattern of the crystalline form XXII of the hydrobromide salt of the compound 1.

FIG. 69 is a TGA diagram of the crystalline form XXII of the hydrobromide salt of the compound 1.

FIG. 70 is a DSC diagram of the crystalline form XXII of the hydrobromide salt of the compound 1.

FIG. 71 is an XRD pattern of the crystalline form XXIII of the mesylate salt of the compound 1.

FIG. 72 is a TGA diagram of the crystalline form XXIII of the compound 1 mesylate salt.

FIG. 73 is a DSC diagram of the crystalline form XXIII of the compound 1 mesylate salt.

FIG. 74 is the XRD pattern of the crystalline form XXIV of the mesylate salt of the compound 1.

FIG. 75 is a TGA diagram of the crystalline form XXIV of the compound 1 mesylate salt.

FIG. 76 is a DSC diagram of the crystalline form XXIV of the compound 1 mesylate salt.

FIG. 77 is an XRD pattern of the crystalline form XXV of the sulfate salt of the compound 1.

FIG. 78 is a TGA diagram of the sulfate salt crystalline form XXV of the compound 1.

FIG. 79 is a DSC diagram of the sulfate salt crystalline form XXV of the compound 1.

FIG. 80 is an XRD pattern of the crystalline form XXVI of the compound 1 sulfate salt.

FIG. 81 is a TGA diagram of the sulfate salt crystalline form XXVI of the compound 1.

FIG. 82 is a DSC diagram of the sulfate salt crystalline form XXVI of the compound 1.

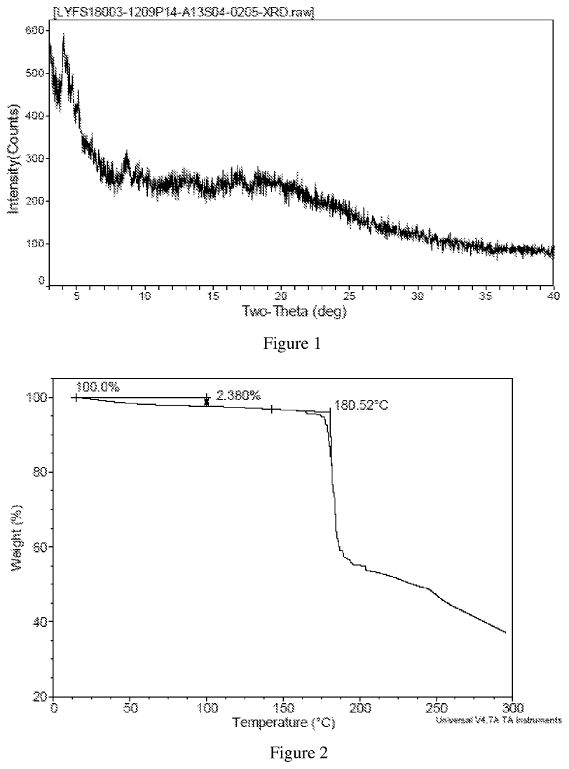

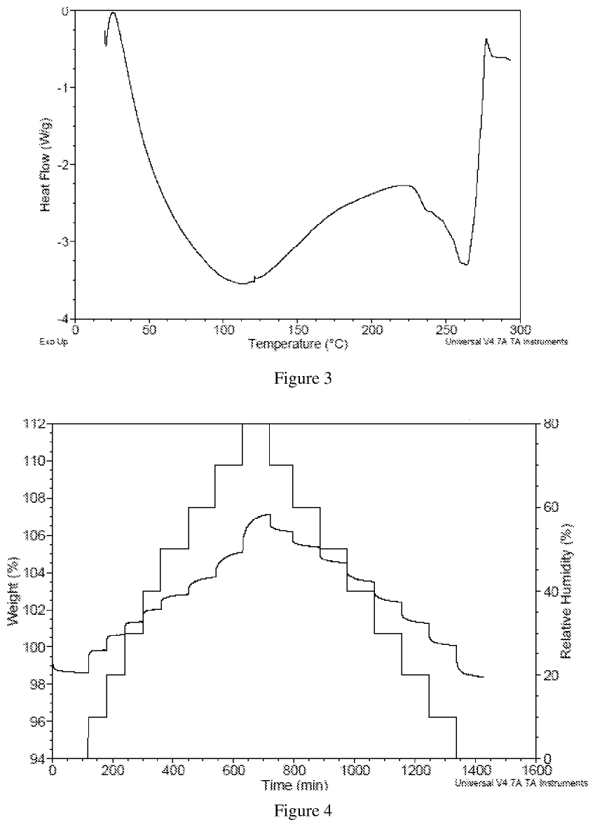

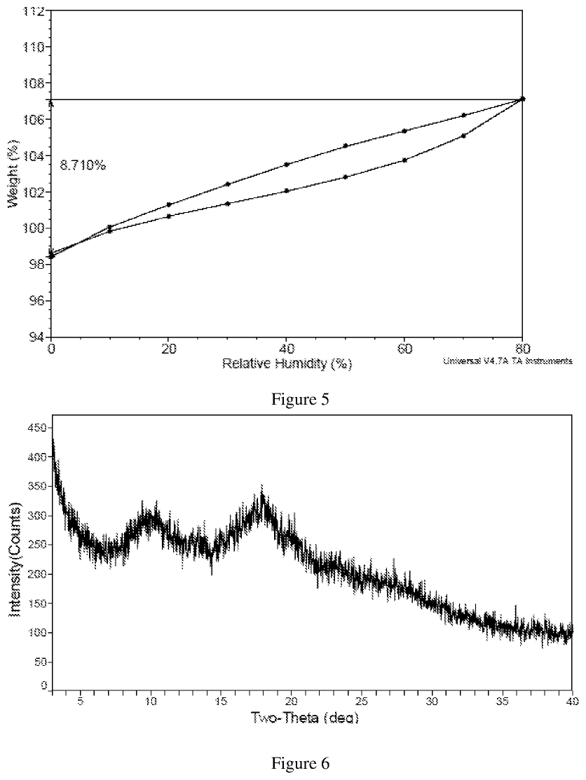

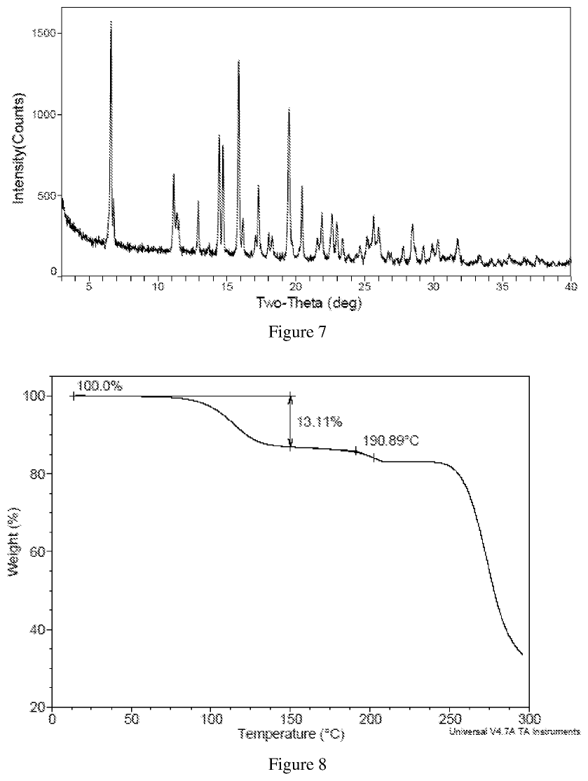

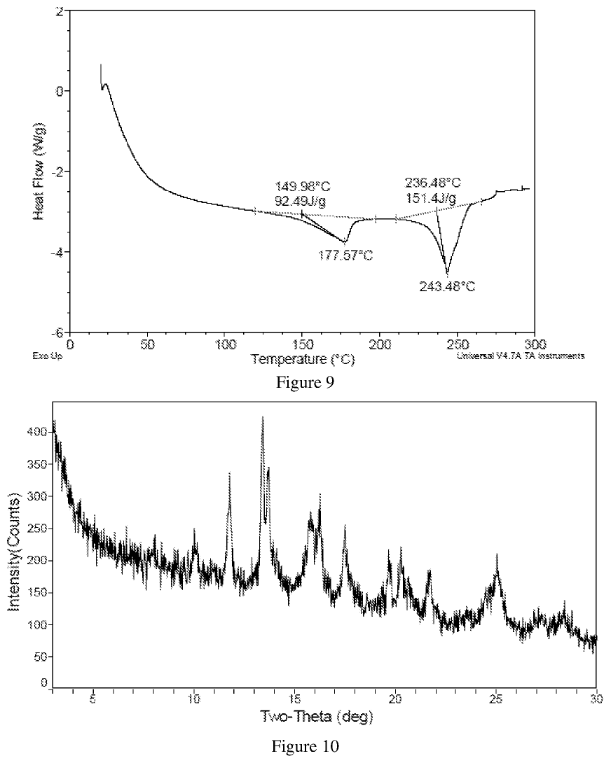

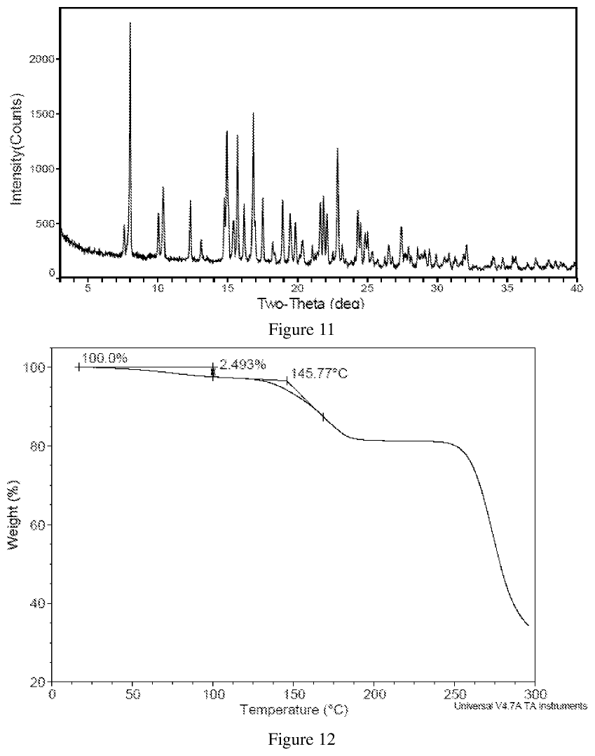

### DETAILED DESCRIPTION OF THE PREFERRED EMBODIMENT

### Examples

In the following examples, the experimental methods are completed in accordance with conventional conditions or conventional test conditions, and the compounds used in the examples are obtained by commercially available or self-made methods.

### Example 1: Preparation of Amorphous Form I of Sulfate of Compound 1

Weigh 100 mg compound 1 and add 0.4 mL isopropanol ultrasonic dissolve, weigh 18 mg of concentrated sulfuric acid (about 1.2 equiv) and dissolve in 0.2 mL isopropanol, add the acid into the sample solution, stir at room temperature overnight, add 3.0 mL of isopropanol and continue stirring for 3 days, the system is emulsion, centrifuge for more than 30 minutes to separate the solid, dry the solid at 50° C. to obtain Amorphous Form I of Sulfate of compound 1.

### Example 2: Preparation of Hydrochloride Amorphous Form II of Compound 1

Weigh 100 mg of compound 1, add 0.4 mL of acetone and perform ultrasonic dissolving, weigh 18 mg of concentrated hydrochloric acid (about 1.2 equiv) and dissolve in 0.2 mL of acetone, add the acid solution into the sample solution, stir at room temperature overnight, the system is viscous, add 3.0 mL of acetone and continue stirring overnight, centrifuge, place the solid at 50° C. overnight to obtain the amorphous form II of compound 1 hydrochloride.

### Example 3: Preparation of Hydrochloride Crystalline Form III of Compound 1

Weigh compound 1 (100 mg), add 1.6 mL of ethyl acetate and heat to 65° C. to dissolve, weigh 19 mg of concentrated hydrochloric acid (about 1.2 equiv) and dissolve in 0.2 mL of ethyl acetate, add acid solution into the sample solution, add 2.0 mL of ethyl acetate and stir at 65° C. for 10 minutes, stop heating, naturally reduce to room temperature, stir for 2 days, centrifuge, dry the solid at 50° C. to obtain compound 1 hydrochloride crystalline form III.

### Example 4: Preparation of Hydrochloride Crystalline Form IV of Compound 1

Weigh compound 1 hydrochloride crystalline form III to desolvation at 180° C. gave anhydrous compound 1 hydrochloride crystalline form IV in poor crystalline state.

### Example 5: Preparation of Maleate Crystalline Form V of Compound 1

Weigh 100 mg compound 1, add 0.8 mL ethyl acetate and heat up to 65° C., weigh 22 mg maleic acid (about 1.2 equiv), dissolve in 0.2 mL ethyl acetate at 65° C., add the acid solution into the sample solution, stir at room temperature for 1 hour, stop heating, stir at room temperature overnight, precipitate a large number of solids, centrifuge, dry the solids at 50° C. to obtain the crystalline form V of compound 1 maleate.

### Example 6: Preparation of Hydrobromide Salt Crystalline Form VI of Compound 1

Weigh 100 mg compound 1, add 0.4 mL acetone and dissolve it in ultrasonic dissolving, weigh 38 mg of 40% hydrobromic acid (about 1.2 equiv) and dissolve it in 0.2 mL acetone, add the acid solution into the sample solution, stir at room temperature overnight and a large amount of turbidity occurs, add 0.4 mL of acetone, continue stirring for 5 hours and then centrifuge, dry the solid at 50° C. to obtain the crystalline form VI of compound hydrobromide salt of compound 1.

### Example 7: Preparation of Methanesulfonate Amorphous Form VII of Compound 1

Weigh 100 mg compound 1, add 0.4 mL isopropanol and sonicate to dissolve, and weigh 22 mg methanesulfonic acid (about 1.2 equiv), add 0.2 mL of isopropanol to dissolve it, add the acid solution into the sample solution, stir at 4° C. for 3 days without solid precipitation, add 1.0 mL isopropyl ether and 0.4 mL isopropanol, the system is largely turbid, stir at room temperature for 6 hours and then centrifuge, dry the solid at 50° C., and obtain the amorphous form VII of compound methanesulfonate.

### Example 8: Preparation of Sodium Salt Amorphous Form VIII of Compound 1

Weigh compound 1 (100 mg), add 0.4 mL ethanol and ultrasonic dissolve, add 7.5 mg sodium hydroxide solid (about 1.2 equiv), stir at room temperature to dissolve, stir overnight without solid precipitation, add 2.0 mL isopropyl ether and a large amount of solid precipitation, continue stirring overnight and then centrifuge, dry the solid at 50° C., and obtain compound 1 sodium salt amorphous form VIII.

### Example 9: Preparation of Potassium Salt Amorphous Form IX of Compound 1

Weigh compound 1 (100 mg), add 0.4 mL ethanol and ultrasonic dissolve, add 13 mg potassium hydroxide solid (about 1.2 equiv), stir and dissolve at room temperature, stir overnight without solid precipitation, add 2.0 mL isopropyl ether, stir at room temperature overnight, if solid precipitation occurs, add 2.0 mL isopropyl ether and continue stirring for 3 hours, then centrifuge, dry the solid at 50° C. to obtain compound 1 amorphous form IX of potassium salt.

### Example 10: Preparation of the Crystalline Form X of the Compound 1

weigh compound 1 (100 mg), added with 2.0 mL of isopropyl acetate and stirred at 4° C. for 4 days, and air dried at room temperature to obtain formula 1 compound crystalline form X.

### Example 11: Preparation of the Monohydrate Crystalline Form XI of Compound 1

Weigh 100 mg of compound 1, add 2.0 mL of isopropyl ether, stir at 4° C. for 4 days, and dry at room temperature to obtain the crystalline form XI of compound 1 monohydrate.

### Example 12: Preparation of the Di-Trifluoroethanol Crystalline Form XII of Compound 1

Weigh 100 mg of compound 1 and place it in a vial containing 5.0 mL of trifluoroethanol at room temperature for 7 days to give compound 1 XII as a crystalline form of di-trifluoroethanol.

### Example 13: Preparation of the Semi-Dimethylsulfoxide Solvate Crystalline Form XIII of the Compound 1

Weigh 100 mg of compound 1, add 0.6 mL acetonitrile and 0.3 mL dimethylsulfoxide, place the crystal pulp at 40° C. for 1 day, and dry the solid at room temperature to obtain the semi-dimethylsulfoxide solvate crystalline form XIII of compound 1.

### Example 14: Preparation of the Semi-Methylcyclohexane Solvate Crystalline Form XIV of the Compound 1

Weigh 100 mg compound 1, add 1.0 mL ethyl acetate and perform ultrasonic dissolving, then add the clear liquid dropwise into 10.0 mL of methylcyclohexane, precipitate solid immediately, continue stirring for 5 minutes, and then centrifuge to obtain the semi-methylcyclohexane solvate crystalline form XIV of the compound 1.

### Example 15: Preparation of the Semi-Tetrahydrofuran Solvate Crystalline Form XV of the Compound 1

Compound 1 (50 mg) was weighed and allowed to stand at room temperature for 3 days in a bottle containing 3.0 mL of tetrahydrofuran to obtain the semi-tetrahydrofuran solvate crystalline form XV of the compound 1.

### Example 16: Preparation of the Amorphous Form XVI of Compound 1

Compound (50 mg) was weighed and added with 0.2 mL of methanol and allowed to stand at room temperature for 3 days to obtain the amorphous form of formula 1 compound XVI.

### Example 17: Preparation of Crystalline Form XVII of the Compound 1

Weigh 100 mg compound 1 into a 20 mL glass bottle, add 9.5 mL of pure acetonitrile, shake for 10 s, gradually dissolve the compound, and allow to stand for a period of time to precipitate a large amount of solid. Stir with a stirrer bar overnight and centrifuge, discarding the supernatant, to give the crystalline form XVII of the compound 1.

### Example 18: Preparation of Hydrochloride Crystalline Form XVIII of Compound 1

Weigh 40-50 mg hydrochloride amorphous form II of compound 1 into a 4 mL glass bottle, add a stirrer, add 500 μl of tetrahydrofuran, stir the mixture at 40° C. for 6 min, perform rapid centrifugation, take the residual solid and dry it in a vacuum drying oven (−0.1 MPa, 25° C.), to obtain Hydrochloride Crystalline Form XVIII of Compound 1.

### Example 19: Preparation of the Hydrobromide Salt Amorphous Form XIX of the Compound 1

Weigh 1.0 g compound 1 into a 40 mL glass bottle, add 10 mL of acetone to dissolve it, then add 230.6 mg of hydrobromic acid (dilute with 2 mL of acetone), no precipitation occurs after overnight stirring, add 10 mL of anti-solvent ethyl acetate to precipitate solid, continue stirring the sample solution for 1 day, perform rapid centrifugation, place the residual solid under vacuum (−0.1 MPa, 40° C.) to dry, and get the hydrobromide salt amorphous form XIX of the compound 1.

### Example 20: Preparation of the Hydrobromide Crystalline Form XX of Compound 1

Weigh 40-50 mg of the hydrobromide salt amorphous form XIX of compound 1 into a 4 mL glass bottle, add a stirrer, add 500 μl of methanol, stir the mixture at 40° C. for 6 days, perform rapid centrifugation, take the residual solid and dry it in a vacuum drying oven (−0.1 MPa, 25° C.) to obtain the Hydrobromide Crystalline Form XX of Compound 1.

### Example 21: Preparation of the Hydrobromide Crystalline Form XXI of Compound 1

Weigh 40-50 mg of the hydrobromide salt amorphous form XIX of compound 1 into a 4 mL glass bottle, add a stirrer, add 500 μl of acetonitrile, the obtained mixture is stirred at 40° C. for 6 days, perform rapid centrifugation, take the residual solid and dry it in a vacuum drying oven (−0.1 MPa, 25° C.) to obtain hydrobromide crystalline form XXI of compound 1.

### Example 22: Preparation of the Hydrobromide Crystalline Form XXII of Compound 1

Weigh 40-50 mg of the hydrobromide salt amorphous form XIX of compound 1 into a 4 mL glass bottle, add a stirrer, then add 500 μl of tetrahydrofuran, the obtained mixture is stirred at 40° C. for 6 days, perform rapid centrifugation, take the residual solid and dry it in a vacuum drying oven (−0.1 MPa, 25° C.) to obtain hydrobromide crystalline form XXII of compound 1.

### Example 23: Preparation of Methanesulfonate Crystalline Form XXIII of Compound 1

Weigh 40-50 mg of the methanesulfonate amorphous form VII of compound 1 into a 4 mL glass bottle, add a stirrer, then add 500 μl ethanol, the obtained mixture is stirred at 40° C. for 6 days, make rapid centrifugation, take the residual solid and dry it in a vacuum drying oven (−0.1 MPa, 25° C.) to obtain the methanesulfonate crystalline form XXIII of compound 1.

### Example 24: Formula 1 Preparation of the Methanesulfonate Crystalline Form XXIV of Compound 1

Weigh 40-50 mg of the methanesulfonate amorphous form VII of compound 1 into a 4 mL glass bottle, add a stirrer, then add 500 μl 1,4-dioxane, the obtained mixture is stirred at 40° C. for 6 days, make rapid centrifugation, take the residual solid and dry it in a vacuum drying oven (−0.1 MPa, 25° C.) to obtain the methanesulfonate crystalline form XXIV of compound 1.

### Example 25: Preparation of the Sulfate Crystalline Form XXV of Compound 1

Weigh 40-50 mg of sulfate amorphous form I of compound 1 into a 4 mL glass bottle, add a stirrer, then add 500 μl of methanol, after the solution obtained is volatilized at room temperature, take the residual solid and dry it in a vacuum drying oven (−0.1 MPa, 25° C.) to obtain the Sulfate Crystalline Form XXV of Compound 1.

### Example 26: Preparation of the Sulfate Crystalline Form XXVI of Compound 1

Weigh 40-50 mg of the sulfate amorphous form I of compound 1 into a 4 mL glass bottle, add a stirrer, then add 500 μl of tetrahydrofuran, the obtained mixture is stirred at 40° C. for 3 days, make rapid centrifugation, take the residual solid and dry it in a vacuum drying oven (−0.1 MPa, 25° C.), and obtain the Sulfate Crystalline Form XXVI of Compound 1.

### Example 27: Identification and Characterization of the Form I-XXVI of Compound 1

The instruments used and their parameters are as follows:
XPRD—X-ray powder diffraction, using Bruker D8 Advance Diffractometer to characterize solids. Copper target wavelength is 1.54 Å Kα radiation (40 kV, 40 mA), 0-20 goniometer, Mo monochromator, Lynxeye detector, detection angle is 3-40° 2θ/3-30° 20, step size It is 0.02° 20, the speed is 0.2 s/step, and the detection sample weight is >2 mg.
TGA—Thermo gravimetric analysis, using TA Instruments Q500 TGA, the detection sample size is 1 mg-10 mg, the common detection method is Hi-Res sensitivity 3.0, Ramp 10.00° C./min, res 5.0 to 150.00° C., Ramp 10.00° C./min to 350° C.
DSC—differential scanning calorimetry analysis, using TA Instruments Q200 DSC, the detection sample weight is 0.5 mg-5 mg, the gas flow rate is 40 mL/min, the common detection method is Equilibrate, 20° C., Ramp 10° C./min to 280° C.-300° C.
DVS—Dynamic Vapour Sorption analysis, the detection sample weight is 1 mg-10 mg, the gas flow rate is 10 mL/min, the common detection method is equilibrium at 25° C., humidity 0%, isothermal for 90 minutes, if the weight percentage is less than 0.0100, the next isothermal test is aborted for 15.00 minutes, and the 10% step humidity is 80.00% every 90 minutes. If the weight percentage is less than 0.0100, the next isothermal test is aborted for 15.00 minutes, and the step humidity is 10% to 0.00% every 90 minutes.
For the above identification and characterization results of XPRD, TGA, DSC, and DVS, please refer to FIG. 1-82, Table 1-15 and related description.

### Example 28: Competitive Experiment of Crystalline Form X and Crystalline Form XI

Take an equal amount of crystalline form X and crystalline form XI samples, mix them uniformly, and sample for XRD detection. Divide the above sample into three equally, add acetone/n-heptane (the volume ratio is 1/3 v:v), dichloromethane/n-heptane (the volume ratio is 1/3 v:v) and acetone respectively/Water (volume ratio of 1/3 v:v) mixed solvent to form a suspension, stirred at room temperature for 1-3 days, centrifuged to sample for XRD detection, the results showed that the mixed sample of crystalline form X and crystalline form XI Stirring in the three systems all converted to crystalline form XI. The most stable form at room temperature is crystalline form XI (detection of environmental humidity 46% RH-52% RH).

### Example 29: Room Temperature Volatile Crystallization Experiment

Take about 5 mg of the compound 1, add the corresponding solvent to obtain a clear solution, and place it at room temperature to evaporate to dryness. The obtained solid was characterized by XPRD. Specific experiments and results are shown in Table 16 below.

TABLE 16

| | | Solvent 1/ | |
|---|---|---|---|
| | | Solvent 2 | Characterization |
| Solvent 1 | Solvent 2 | (mL) | results |
| Methanol | | 0.2 | Form XVI |
| Ethanol | | 0.2 | Form XVI |
| Acetone | | 0.2 | Form XVI |
| Ethyl acetate | | 0.2 | Form XI |
| Tetrahydrofuran | | 0.2 | Form XVI |
| Chloroform | | 0.2 | Form XVI |
| Methanol | Water | 1.4/0.2 | Form XI |
| Ethanol | Water | 1.0/0.1 | Form XI |
| Tri fluoroethanol | Water | 0.6/0.1 | Form XI |

### Example 30: High-Temperature Volatile Crystallization Experiment

Take about 5 mg of the compound 1, add the corresponding solvent to obtain a clear solution, and place it at 40° C. to evaporate to dryness. The obtained solid was characterized by XPRD. Specific experiments and results are shown in Table 17 below.

TABLE 17

| | | Solvent 1/ | |
|---|---|---|---|
| | | Solvent 2 | Characterization |
| Solvent 1 | Solvent 2 | (mL) | results |
| Isopropanol | | 0.2 | Form XVI |
| Dimethyl sulfoxide | | 0.2 | Form XVI |
| Methanol | Water | 1.0/0.1 | Form XI |
| Ethanol | Water | 2.0/0.1 | Form XVI |
| Acetone | 2-Butanol | 0.1/0.1 | Form XVI |
| Tetrahydrofuran | n-Heptane | 0.1/0.1 | Form XVI |
| Dichloromethane | 1,4-Dioxane | 0.2/0.1 | Form XVI |

### Example 31: Mixed Solvent Crystallization Experiment

Take about 15 mg of the compound 1, add solvent 1 to obtain a clear solution, and slowly add solvent 2 under stirring. After the solid precipitated, the stirring was continued for 5 minutes, and samples were taken for XPRD characterization. If there is no solid precipitation, an oily substance is obtained, or the characterization result is an amorphous form, the stirring is continued overnight, and the XPRD characterization is performed the next day. The specific experiments and results are shown in Table 18 below.

TABLE 18

| | | Solvent 1/ | |
|---|---|---|---|
| | | Solvent 2 | Characterization |
| Solvent 1 | Solvent 2 | (mL) | results |
| Methanol | Water | 0.2/0.2 | Form XI |
| Ethanol | Water | 0.4/0.4 | Form XI |
| Trifluoroethanol | Water | 0.4/0.2 | Form XVI |
| Isopropanol | Water | 0.4/0.6 | Form XI |
| Acetone | Water | 0.3/0.4 | Form XI |
| Tetrahydrofuran | Water | 0.2/0.4 | Form XVI |
| 1,4-Dioxane | Water | 0.2/0.6 | Form XI |
| Acetonitrile | Water | 1.5/1.0 | Form XI |
| Dimethyl sulfoxide | Water | 0.2/0.4 | Form XVI |
| Acetone | n-Heptane | 0.2/2.0 | Form XI |

### Example 32: Heating and Cooling Crystallization Experiment

Take about 15 mg of the compound 1 and add a solvent at 50° C.-60° C. to obtain a clear solution. After keeping the temperature for 5 minutes, place it in an ice-salt bath and stir. After the solid is precipitated, it is centrifuged immediately, and a solid sample is taken for XRD characterization. Specific experiments and results are shown in Table 19 below.

TABLE 19

| | | Solvent 1/ | |
|---|---|---|---|
| | | Solvent 2 | Characterization |
| Solvent 1 | Solvent 2 | (mL) | results |
| Methanol | | 0.1 | Form XI |
| Ethanol | | 0.1 | Form XI |
| Isopropanol | | 0.1 | Form XVI |
| Ethyl acetate | | 0.1 | Form XI |
| Methanol | Water | 0.8/0.1 | Form XI |
| Ethanol | Water | 1.0/0.3 | Form XI |
| Trifluoroethanol | Water | 0.4/0.1 | Form XI |
| Isopropanol | Water | 0.4/0.1 | Form XI |
| Acetone | Water | 0.8/0.3 | Form XI |
| Tetrahydrofuran | Water | 0.4/0.2 | Form XI |

### Example 33: Low-Temperature Slurry Crystallization

About 15 mg of the compound 1 was added to the corresponding solvent to obtain a suspension, stirred at 4° C. for 3 hours and 7 days, the suspension was centrifuged, and the solid was taken for XRD characterization. The specific experiments and results are shown in Table 20 below.

TABLE 20

| | | Solvent 1/ | |
|---|---|---|---|
| | | Solvent 2 | Characterization |
| Solvent 1 | Solvent 2 | (mL) | results (3 h/7 d) |
| Isopropyl ether | | 0.4 | Form XI/Form XI |
| Acetonitrile | | 0.4 | Form XI/Form XI |
| Nitromethane | Methanol | 0.4/0.1 | Form XI/Form XI |

### Example 34: Room Temperature Slurry Crystallization

Take about 15 mg of the compound 1, add the corresponding solvent to obtain a suspension, and stir at room temperature for 3 hours and 7 days. The suspension after taking the crystal slurry was centrifuged, and the solid was taken for XRD characterization. The specific experiments and results are shown in Table 21 below.

TABLE 21

| | | Solvent 1/ | Characterization |
|---|---|---|---|
| Solvent 1 | Solvent 2 | Solvent 2 (mL) | results (3 h/7 d) |
| Water | | 0.4 | Amorphous/Form XI |
| Methanol | Water | 0.1/0.2 | Form XI/Form XI |
| Ethanol | Water | 0.1/0.2 | Form XI/Form XI |
| Trifluoroethanol | Water | 0.1/0.2 | Form XI/Form XI |
| Isopropanol | Water | 0.2/0.2 | Form XI/Form XI |
| Acetone | Water | 0.2/0.2 | Form XI/Form XI |
| Tetrahydrofuran | Water | 0.2/0.4 | Form XI/Form XI |
| 1,4-Dioxane | Water | 0.2/0.4 | Form XI/Form XI |
| Dimethyl | Water | 0.1/0.2 | Amorphous/Form XI |
| sulfoxide | | | |
| N-butanol | n-Heptane | 0.1/0.4 | Form XI/Form XI |
| Butanone | Methyl cy cl ohexane | 0.2/0.4 | Form XI/Form XI |

### Example 35: High Temperature Slurry Crystallization

Take about 15 mg of the compound 1, add the corresponding solvent to obtain a suspension, and stir at high temperature for 3 hours and 7 days. The suspension after taking the crystal slurry was centrifuged, and the solid was taken for XRD characterization. Specific experiments and results are shown in Table 22 below.

TABLE 22

| | | Solvent 1/ | Characterization |
|---|---|---|---|
| Solvent 1 | Solvent 2 | Solvent 2 (mL) | results (3 h/7 d) |
| Dimethyl | Water | 0.2/0.2 | Form XI/Form XI |
| sulfoxide | | | |
| Acetonitrile | Dimethyl | 0.2/0.1 | Form XIII/Form |
| | sulfoxide | | XIII |

### Example 36: Study on Hygroscopicity of Crystalline Form XI

Take about 10 mg of crystalline form XI sample for Dynamic Vapour Sorption (DVS) test. The conclusions are described in Table 23 below.

TABLE 23

| | | XRPD before and after |
|---|---|---|
| Form | Weight Gain (80% RH) | DVS |
| Form XI | 0.01% | Unchanged |

The above data shows that the crystalline form XI is not easy to absorb water during storage, is easy to store, and can extend the shelf life.

### Example 37: Stability Test of Crystalline Form XI (Different Temperature and Humidity)

Place the sample of Form XI under high temperature, high humidity 75% RH conditions, and sample on Day 0, Day 5, Day 10 and Day 30 to investigate its content, related substances and crystal forms. The results are shown in Table 24.

TABLE 24

| | | Total impurity | |
|---|---|---|---|
| Test conditions | content (%) | content (%) | XPRD |
| Day 0 | 99.5 | 0.31 | Form XI |
| Day 5—75% RH | 99.1 | 0.16 | Not detected |
| Day 5—40° C. | 99.6 | 0.21 | Not detected |
| Day 5—60° C. | 99.1 | 0.15 | Not detected |
| Day 10—75% RH | 99.3 | 0.24 | Form XI |
| Day 10—40° C. | 99.1 | 0.25 | Form XI |
| Day 10—60° C. | 100.1 | 0.25 | Form XI |
| Day 30—75% RH | 100.5 | 0.32 | Form XI |
| Day 30—40° C. | 98.9 | 0.33 | Form XI |

The results showed that the content and purity of form XI measured at 5 days, 10 days and 30 days were almost unchanged under high temperature and high humidity conditions, showing good stability.

### Example 38: Hygroscopicity Test for Amorphous Form XVI

About 10 mg of amorphous form XVI sample was taken for dynamic moisture adsorption (DVS) test. The conclusions are described in Table 25 below:

TABLE 25

| | Weight Gain | XRPD |
|---|---|---|
| Form | (80% RH) | before and after DVS |
| Form XVI | 2.32% | Unchanged |

The above results show that the amorphous XVI sample is not easy to absorb water during storage, is easy to preserve, and can have a shelf life.

### Example 39: Stability Testing of Amorphous Form XVI

The amorphous XVI sample was placed at 60° C., with high humidity 90% RH, under the light condition (light condition: 4500 Lux), and sampled on Day 0/5/10 to investigate its content, related substances and crystal form. The results are shown in Table 26.

TABLE 26

| | | Total impurity | |
|---|---|---|---|
| Test conditions | Content (%) | content (%) | XPRD |
| Day 0 | 99.4 | 0.38 | Amorphous |
| Day 5—90% RH | 102.1 | 0.40 | — |
| Day 5—light condition | 102.4 | 0.44 | — |
| Day 5—60° C. | 101.7 | 0.48 | — |
| Day 10—90% RH | 101.1 | 0.40 | Amorphous |
| Day 10—light condition | 100.4 | 0.57 | Amorphous |
| Day 10—60° C. | 99.0 | 0.54 | Amorphous |

### Example 40: Hygroscopicity Test for Hydrobromide Crystalline Form VI and Maleate Crystalline Form V

Dynamic water sorption (DVS) was performed on hydrobromide and maleate crystalline samples. The conclusions are described in Table 27 below:

TABLE 27

| | Weight Gain | XRPD |
|---|---|---|
| Form | (80% RH) | before and after DVS |
| hydrobromide | 5.1% (hygroscopic) | Not detected |
| crystalline form VI | | |
| maleate crystalline | 1.2% (slightly | Unchanged |
| form V | hygroscopic) | |

### Example 41: Polymorph Screening Test for Hydrochloride

Weigh 40-50 mg of compound 1 into a 4 mL glass bottle, add a stirrer, and then respectively add 500 μl of solvent (as shown in Table 28), the obtained mixture is stirred at 40° C. for 6 days, make rapid centrifugation, and take the residual solid to dry in a vacuum drying oven (−0.1 MPa, 25° C.).

TABLE 28

| No. | Solvent | Hydrochloride |
|---|---|---|
| — | Initial form | Amorphous II |
| 1 | Methanol | Amorphous II |
| 2 | Ethanol | Amorphous II |
| 3 | Isopropanol | Amorphous II |
| 4 | Acetonitrile | Amorphous II |
| 5 | Acetone | Form III |
| 6 | Ethyl acetate | Amorphous II |
| 7 | Acetonitrile:water = 1:1 | Form XI |
| 8 | Tetrahydrofuran | Form XVIII |
| 9 | Toluene | Amorphous II |
| 10 | 1,4-Dioxane | Amorphous II |

### Example 42: Polymorph Screening Test for Hydrobromide

Weigh 40-50 mg of the hydrobromide amorphous form XIX of compound 1 into a 4 mL glass bottle, add a stirrer, and then respectively add 500 μl of solvent (as shown in Table 29 below); the obtained mixture is stirred at 40° C. for 6 days, make rapid centrifugation, and take the residual solid to dry in a vacuum drying oven (−0.1 MPa, 25° C.).

TABLE 29

| No. | Solvent | Hydrobromide |
|---|---|---|
| — | Initial form | Amorphous XIX |
| 1 | Methanol | Form XX |
| 2 | Ethanol | Form VI |
| 3 | Isopropanol | Amorphous XIX |
| 4 | Acetonitrile | Form XXI |
| 5 | Acetone | Amorphous XIX |
| 6 | Ethyl acetate | Amorphous XIX |
| 7 | Acetonitrile:water = 1:1 | Form XI |
| 8 | Tetrahydrofuran | Form XXII |
| 9 | Toluene | Amorphous XIX |
| 10 | 1,4-Dioxane | Amorphous XIX |

### Example 43: Polymorph Screening of Maleate

Weigh 40-50 mg of maleate crystalline form V of compound 1 into a 4 mL glass bottle, add a stirrer, and then respectively add 500 μl of solvent (as shown in Table 30 below), the obtained mixture is stirred at 40° C. for 6 days, make rapid centrifugation, and take the residual solid to dry in a vacuum drying oven (−0.1 MPa, 25° C.).

TABLE 30

| No. | Solvent | Maleate |
|---|---|---|
| — | Initial form | Form V |
| 1 | Methanol | Form V |
| 2 | Ethanol | Form V + Form XI |
| 3 | Isopropanol | Form V |
| 4 | Acetonitrile | Form V |
| 5 | Acetone | Form V |
| 6 | Ethyl acetate | Form V |
| 7 | Acetonitrile:water = 1:1 | Form XI |
| 8 | Tetrahydrofuran | Amorphous XVI |
| 9 | Toluene | Form V |
| 10 | 1,4-Dioxane | Form V |

### Example 44: Polymorph Screening for Sodium Salts

Weigh 40-50 mg of the sodium salt amorphous form VIII of compound 1 into a 4 mL glass bottle, add a stirrer, and then respectively add 500 μl of solvent (as shown in Table 31 below), the obtained mixture is stirred at 40° C. for 6 days, make rapid centrifugation, and take the residual solid to dry in a vacuum drying oven (−0.1 MPa, 25° C.).

TABLE 31

| No. | Solvent | Sodium salt |
|---|---|---|
| — | Initial form | Amorphous VIII |
| 1 | Methanol | Amorphous VIII |
| 2 | Ethanol | Amorphous VIII |
| 3 | Isopropanol | Amorphous VIII |
| 4 | Acetonitrile | Amorphous VIII |
| 5 | Acetone | Amorphous VIII |
| 6 | Ethyl acetate | Amorphous VIII |
| 7 | Acetonitrile:water = 1:1 | Form XI |
| 8 | Tetrahydrofuran | Amorphous VIII |
| 9 | Toluene | Amorphous VIII |
| 10 | 1,4-Dioxane | Amorphous VIII |

### Example 45: Polymorph Screening of Methanesulfonate

Weigh 40-50 mg of the methanesulfonate amorphous form VII of the compound 1 into a 4 mL glass bottle, add a stirrer, and then add 500 μl of solvent (as shown in Table 32 below), respectively. The obtained mixture is stirred at 40° C. for 6 days, quickly centrifuged, and the residual solid is dried in a vacuum drying oven (−0.1 MPa, 25° C.).

TABLE 32

| No. | Solvent | Methanesulfonate |
|---|---|---|
| — | Initial form | Amorphous VII |
| 1 | Methanol | Form XXIII |
| 2 | Ethanol | Form XXIII |
| 3 | Isopropanol | Amorphous VII |
| 4 | Acetonitrile | Amorphous VII |
| 5 | Acetone | Amorphous VII |
| 6 | Ethyl acetate | Amorphous VII |
| 7 | Acetonitrile:water = 1:1 | Amorphous VII |
| 8 | Tetrahydrofuran | Amorphous VII |
| 9 | Toluene | Amorphous VII |
| 10 | 1,4-Dioxane | Form XXIV |

### Example 46: Polymorph Screening of Potassium Salt

Weigh 40-50 mg of potassium salt Amorphous Form IX of compound 1 into a 4 mL glass bottle, add a stirrer, then respectively add 500 μl of solvent (as shown in Table 33 below), the obtained mixture is stirred at 40° C. for 3 days, make rapid centrifugation, and take the residual solid to dry in a vacuum drying oven (−0.1 MPa, 25° C.).

TABLE 33

| No. | Solvent | Potassium salt |
|---|---|---|
| — | Initial form | Amorphous IX |
| 1 | Methanol | Amorphous IX |
| 2 | Ethanol | Amorphous IX |
| 3 | Isopropanol | Amorphous IX |
| 4 | Acetonitrile | Amorphous IX |
| 5 | Acetone | Amorphous IX |
| 6 | Ethyl acetate | Amorphous IX |
| 7 | Tetrahydrofuran | Amorphous IX |
| 8 | Toluene | Amorphous IX |
| 9 | 1,4-Dioxane | Amorphous IX |

### Example 47: Polymorph Screening of Sulfate

Weigh 40-50 mg of sulfate Amorphous Form I of compound 1 into a 4 mL glass bottle, add a stirrer, then respectively add 500 μl of solvent (as shown in Table 34 below), the obtained mixture is stirred at 40° C. for 3 days, make rapid centrifugation, and take the residual solid to dry in a vacuum drying oven (−0.1 MPa, 25° C.).

TABLE 34

| No. | Solvent | Sulfate |
|---|---|---|
| — | Initial form | Amorphous 1 |
| 1 | Methanol | Form XXV |
| 2 | Ethanol | Amorphous 1 |
| 3 | Isopropanol | Amorphous I |
| 4 | Acetonitrile | Amorphous I |
| 5 | Acetone | Amorphous I |
| 6 | Ethyl acetate | Amorphous I |
| 7 | Acetonitrile: water = 1:1 | Amorphous I |
| 8 | Tetrahydrofuran | Form XXVI |
| 9 | Toluene | Amorphous I |
| 10 | 1,4-Dioxane | Form XXVI |

### Example 48: Stability Test for Salt Form Screening

Weigh 30 mg of compound (maleate crystalline form V) into a 8 mL glass bottle, then place it at high temperature (60° C., open), high humidity (room temperature/75% RH, open) and light (room temperature, white light: 6980 lux, ultraviolet 282 μW/cm2), take samples on Day 5/10/30 respectively for testing (HPLC, XRD).

TABLE 35

| | | Total impurity | |
|---|---|---|---|
| Sample | Test conditions | content (%) | XPRD |
| Maleate | Day 0 | 1.20 | Form V |
| crystalline | Day 5—60° C. | 1.20 | Form V |
| form V | Day 5—75% RH | 1.10 | Form V |
| | Day 5—light | 1.10 | Form V |
| | Day 10—60° C. | 1.20 | Form V |
| | Day 10—75% RH | 1.20 | Form V |
| | Day 10—light | 1.10 | Form V |
| | Day 30—60° C. | 1.20 | Form V |
| | Day 30—75% RH | 1.10 | Form V |

The stability results showed that the content and purity of the maleate crystalline form V were almost unchanged on Day 5/10/30 under high temperature, high humidity and light, respectively, showing good stability.

Each reference, including all patents, patent applications and publications referenced in this application, is incorporated herein by reference in its entirety as if each of them were incorporated separately. In addition, it is understood that in the teaching of the present invention, the technicians in the art may make certain changes or modifications to the present invention and that these equivalents will remain within the scope of the present invention as limited by the claims appended to the application.

1. An amorphous or crystalline form of compound 1 or a salt or solvate thereof:

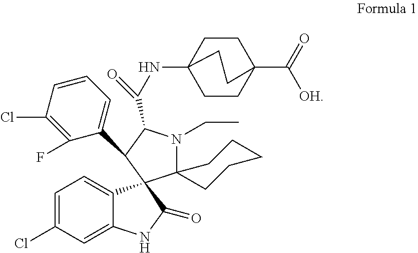

2.-7. (canceled)

8. The form according to claim 1, which is crystalline form V of the maleate salt of the compound 1, comprising one or more characteristic peak positions as measured by XRPD and represented by 2θ angles: 8.159±0.2°, 10.519±0.2°, 15.078±0.2°, 15.839±0.2°, 16.959±0.2% and 22.997±0.2°.

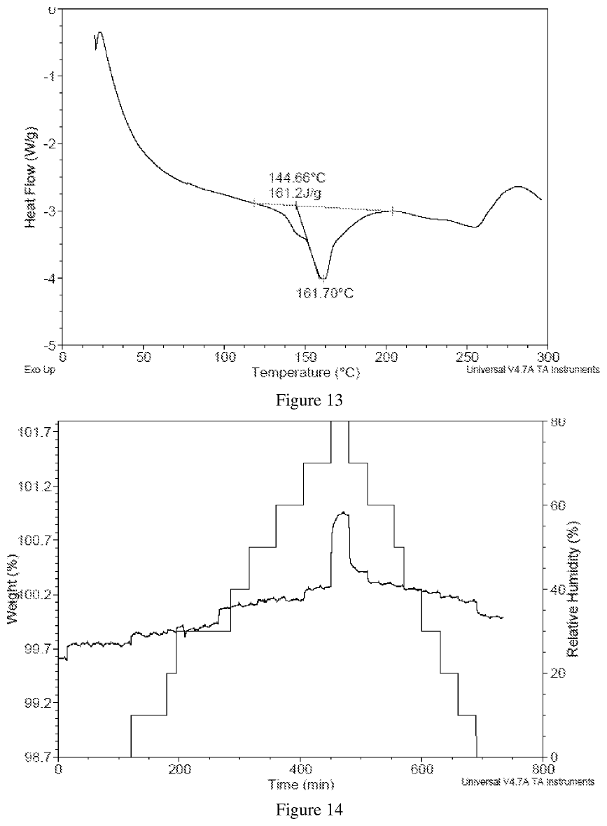

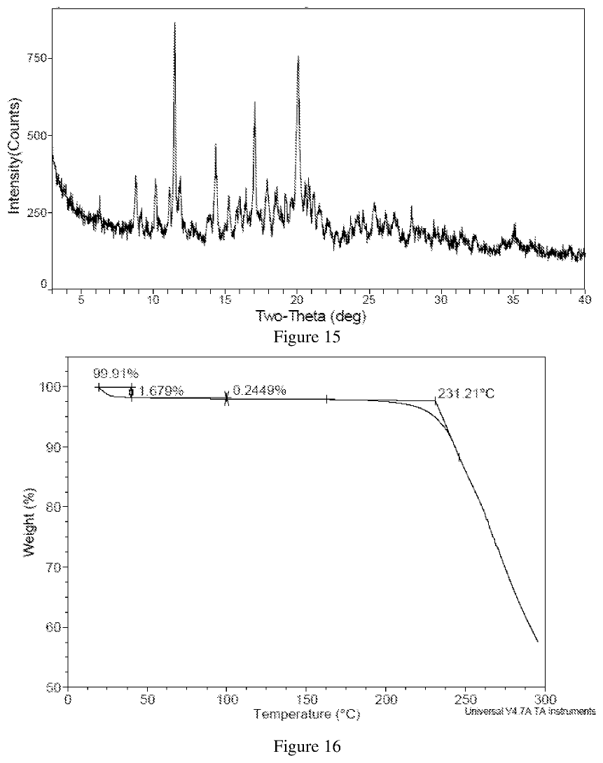

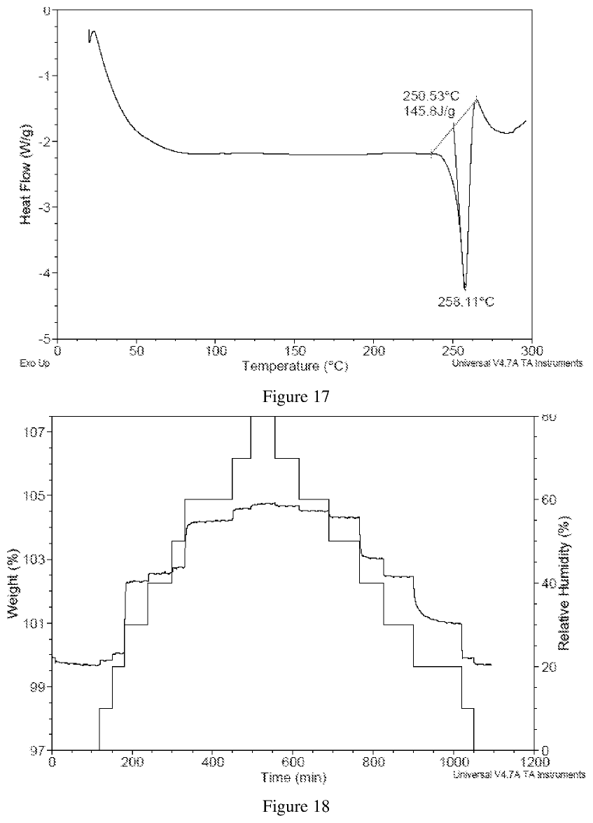

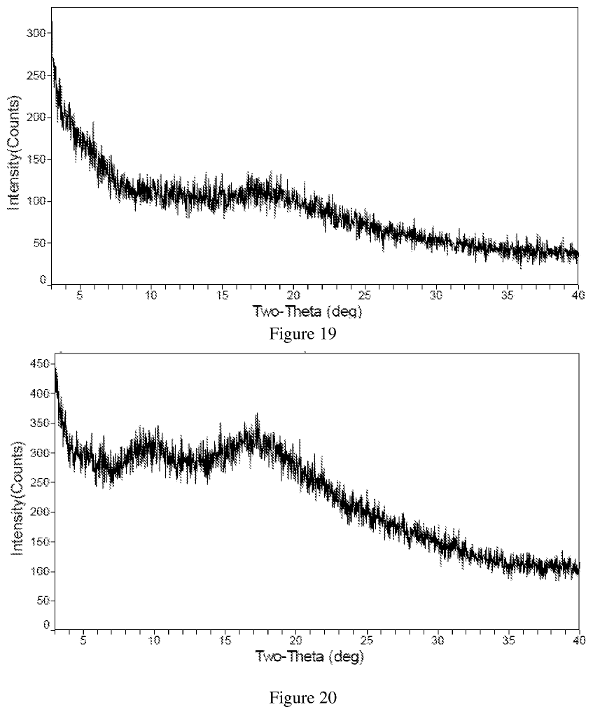

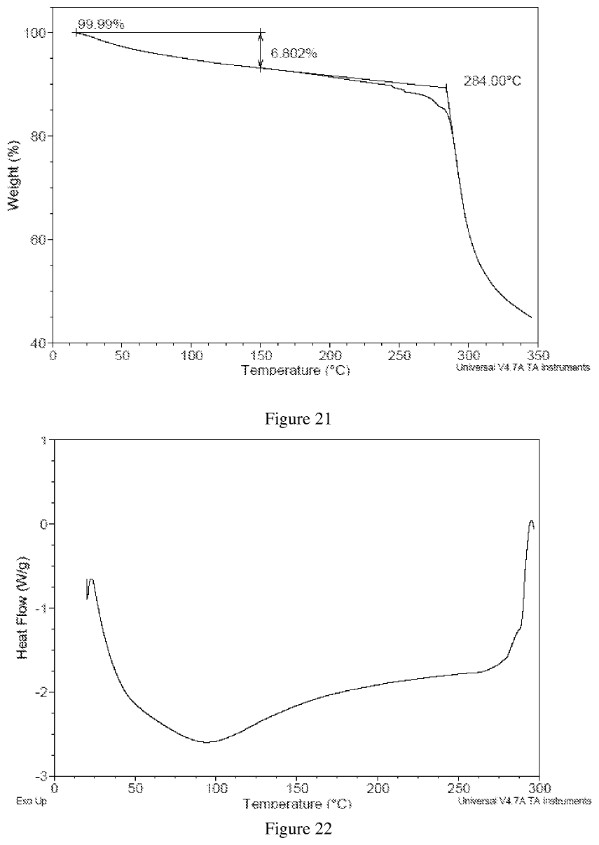

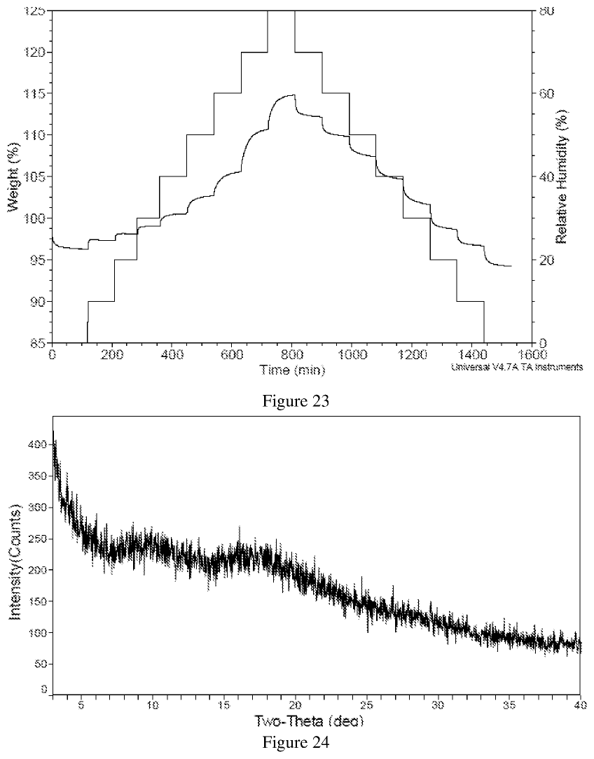

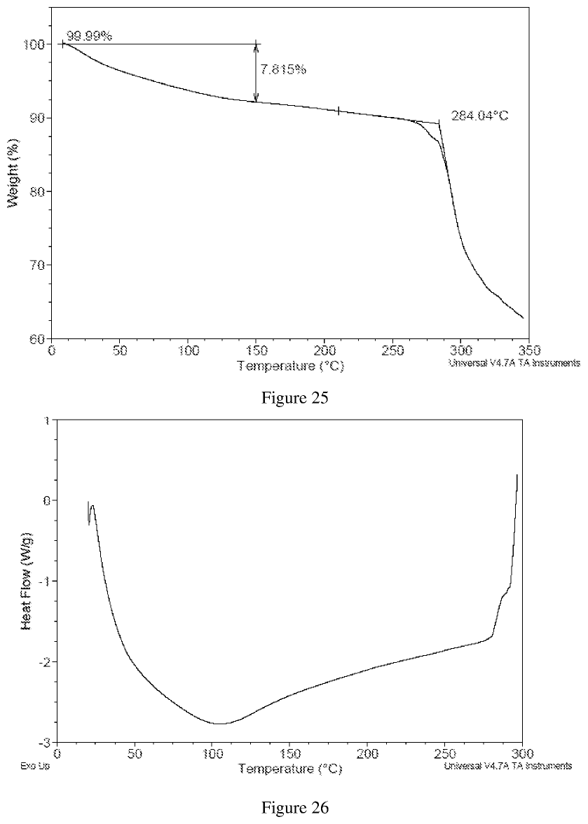

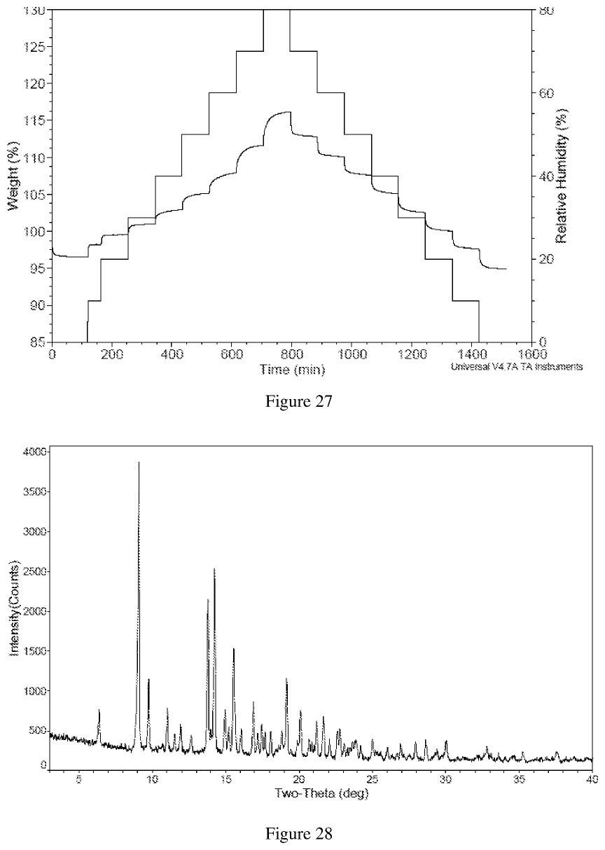

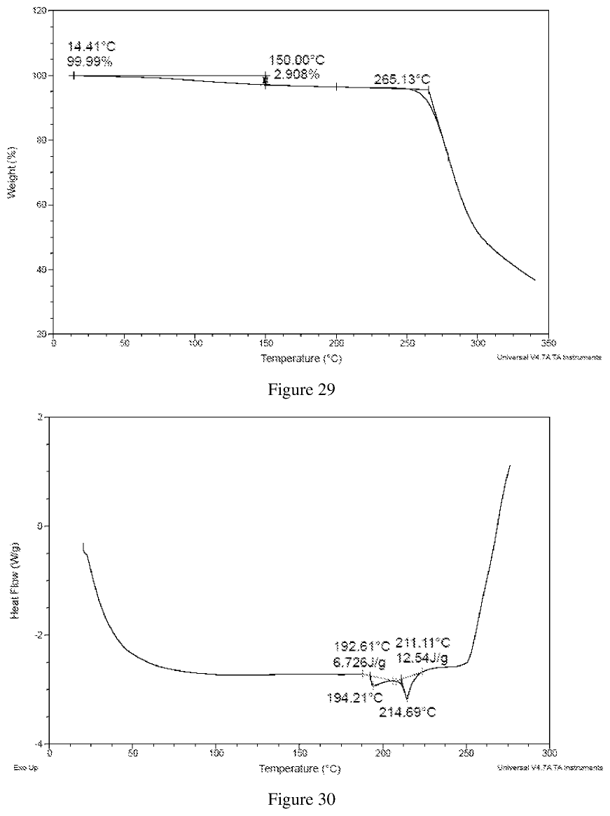

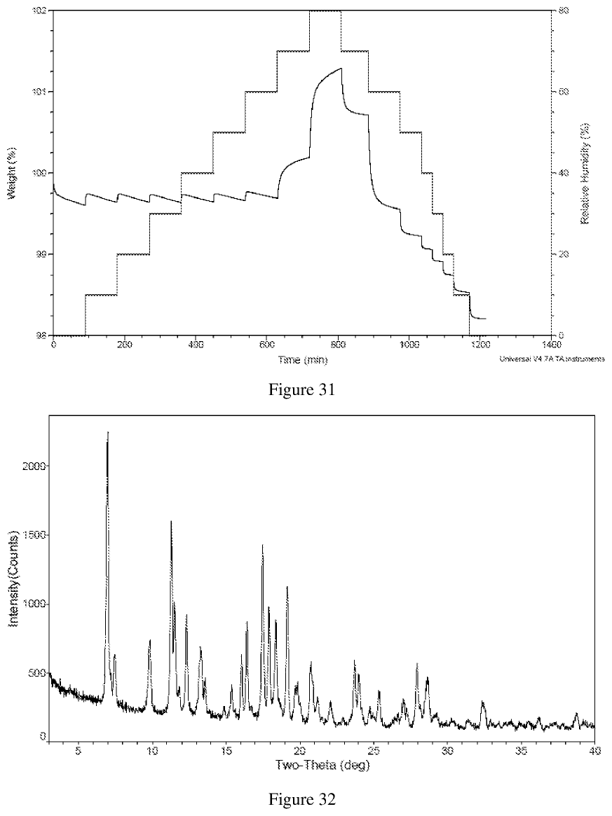

9. The form according to claim 8, comprising one or more of:
1) the XRPD diagram as shown in FIG. 11;
2) the TGA diagram as shown in FIG. 12; and
3) the DSC diagram as shown in FIG. 13.

10.-20. (canceled)

21. The form according to claim 1, which is crystalline form XI of the compound 1 monohydrate, comprising one or more characteristic peak positions as measured by XRPD and represented by 2θ angles: 6.999±0.2°, 11.319±0.2°, 11.522±0.2% and 17.485±0.2°.

22. The form according to claim 21, further comprising one or more additional characteristic peaks positions in the XRPD diagram represented by 2θ angles: 9.858±0.2°, 11.319±0.2°, 11.522±0.2°, 12.341±0.2°, 13.282±0.2°, 17.923±0.2°, 19.159±0.2% and 28.644±0.2°.

23. The form according to claim 21, comprising one or more of XRPD characteristic peaks at positions substantially as shown in Table 2 and an XRPD pattern substantially as shown in FIG. 32.

24. The form according to claim 21, further comprising one or more of:
1) as measured by TGA, a weight loss of 2.4±0.5% by weight before 100° C., and a decomposition temperature of 262±2° C.; and
2) as measured by DSC, a broad endothermic peak at 90° C.-140° C., a melting point of 243±3° C., and decomposition after melting.

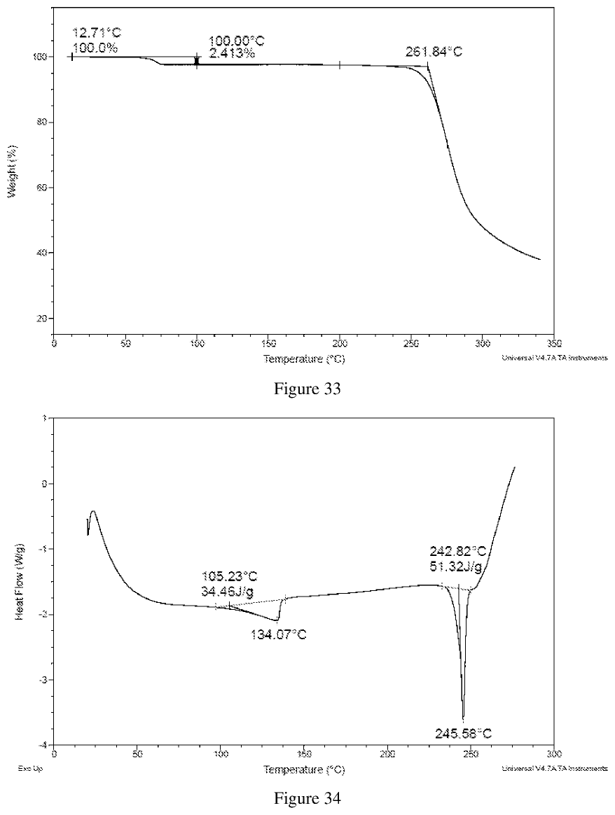

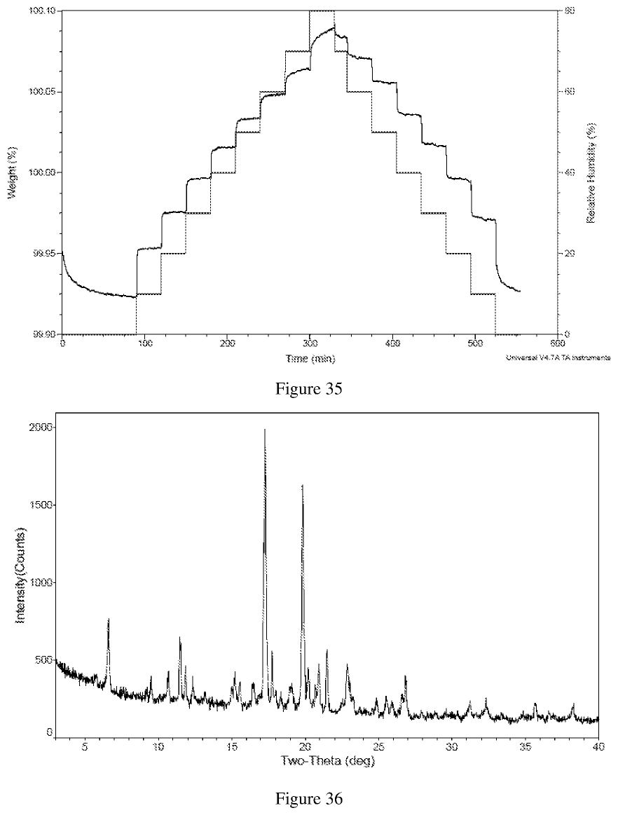

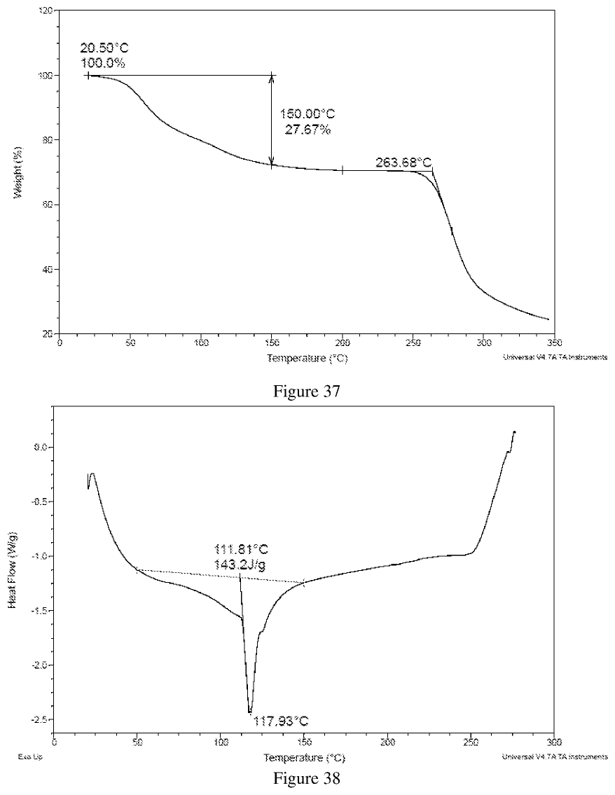

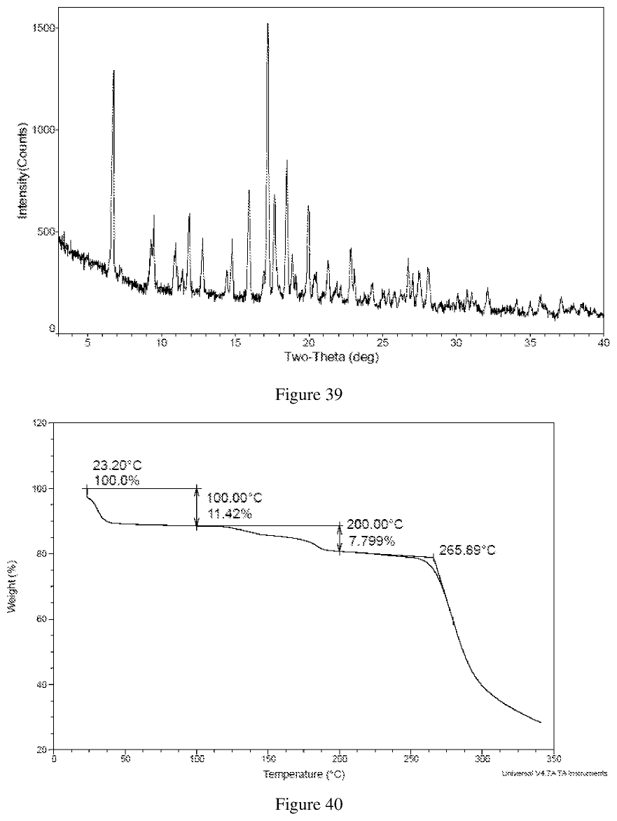

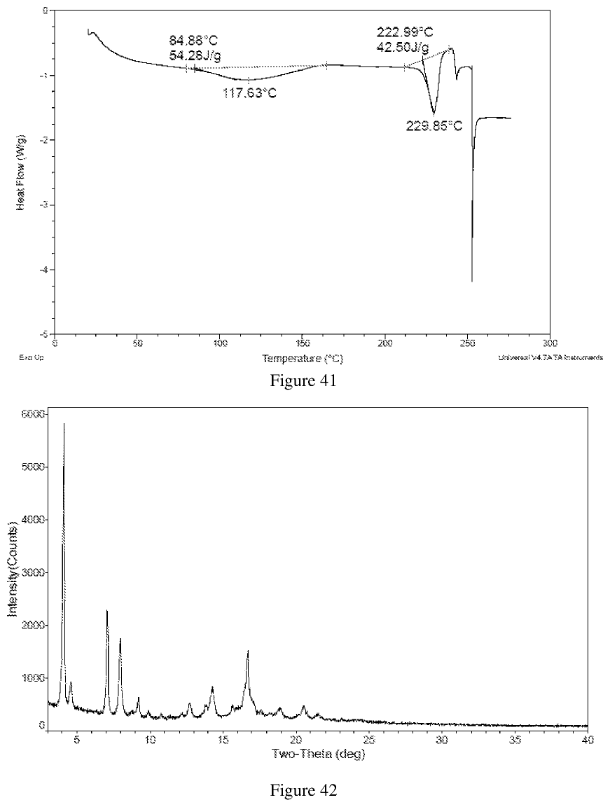

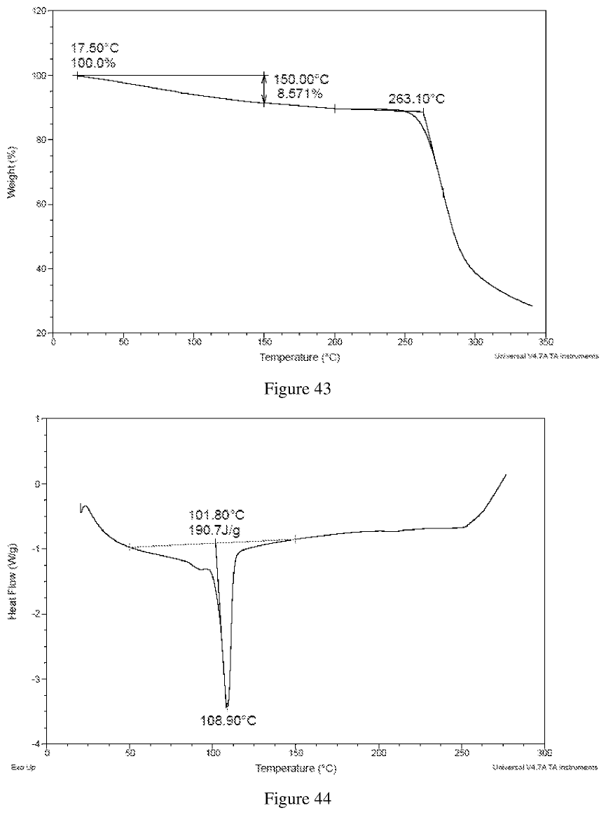

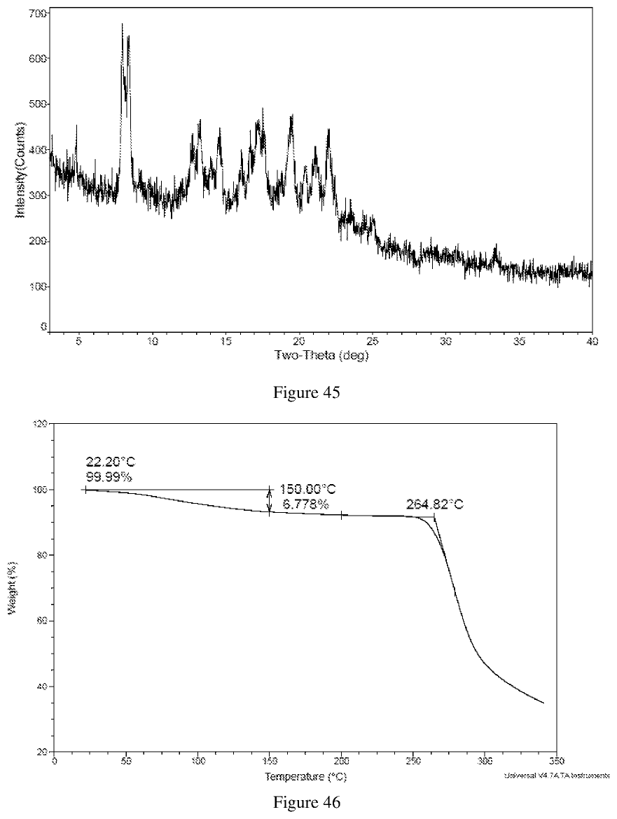

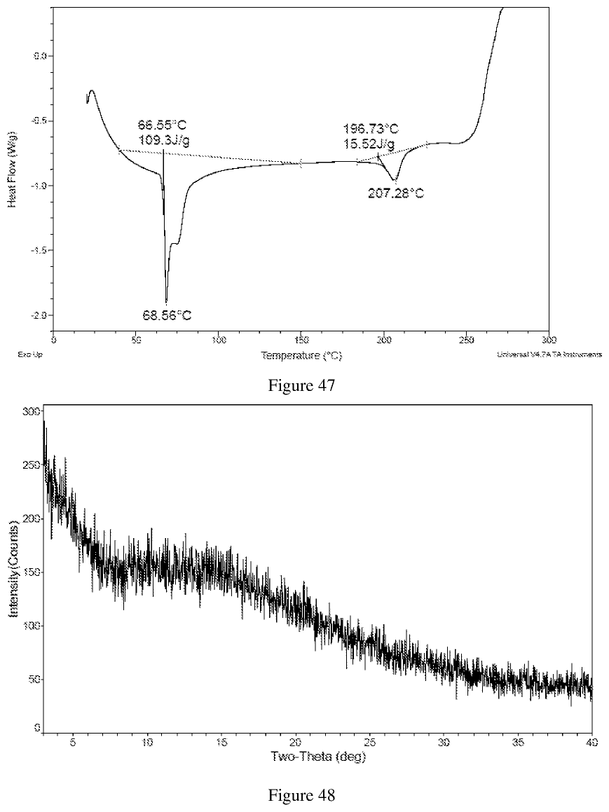

25. The form according to claim 21, further comprising one or more of:
1) the TGA diagram as shown in FIG. 33; and
2) the DSC diagram as shown in FIG. 34.

26.-41. (canceled)

42. The form according to claim 1, which is the compound 1 in amorphous form XVI

43. (canceled)

44. The form according to claim 42, further comprising one or more of:
1) as measured by TGA, a slow weight loss of 2.9±0.1% by weight before 150° C., and a decomposition temperature of 265±2° C.; and
2) no melting peak as measured by DSC.

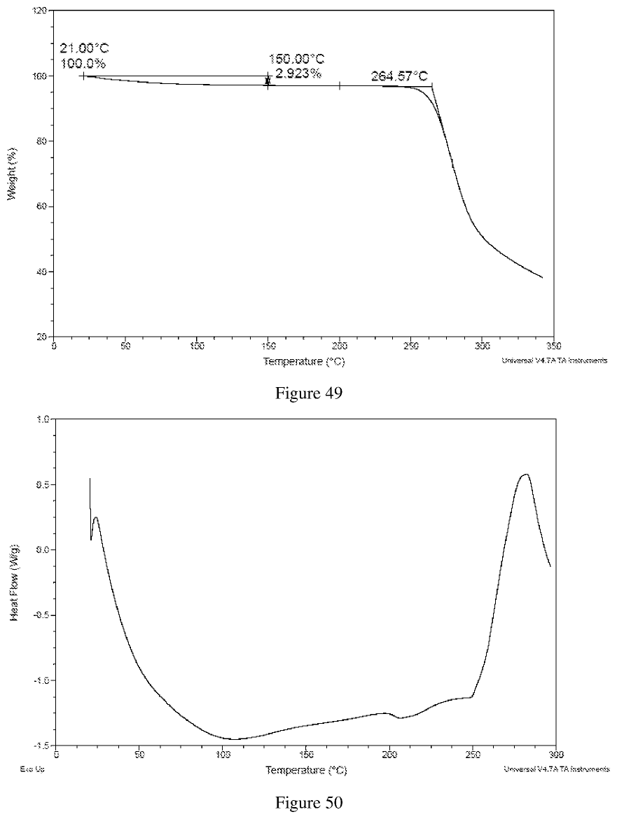

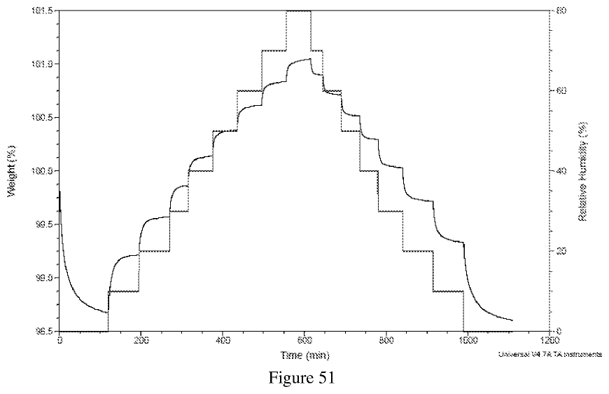

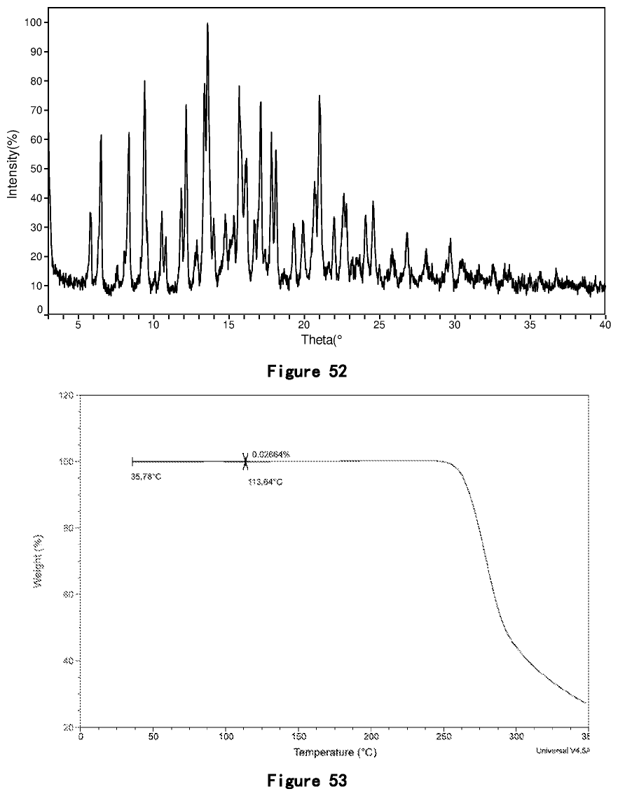

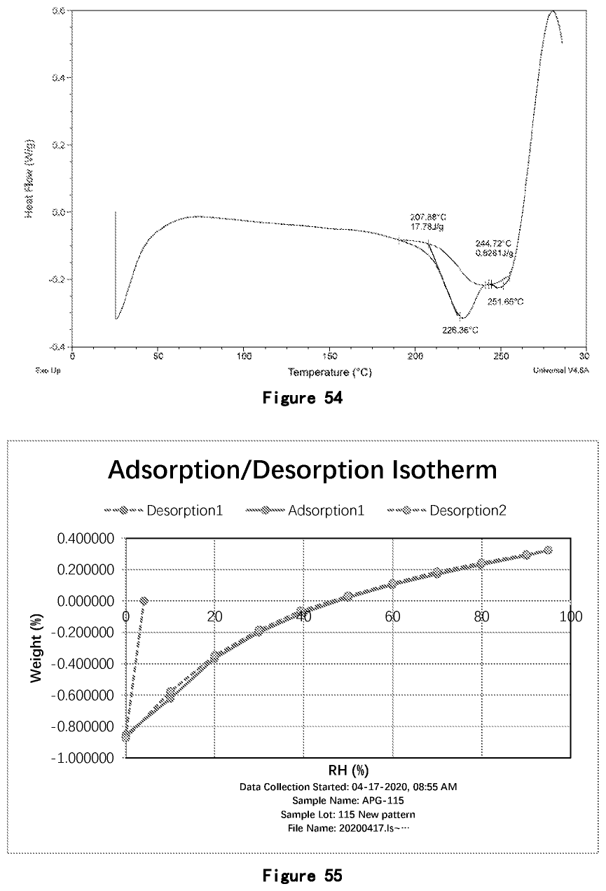

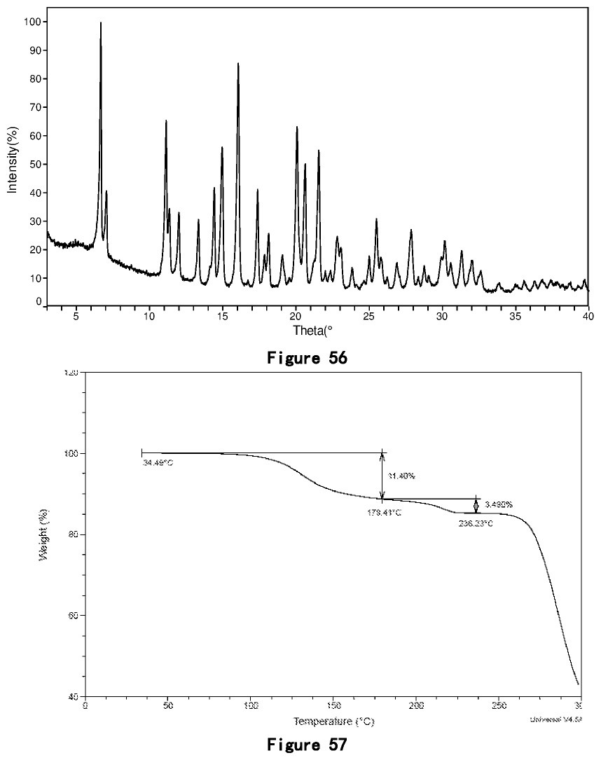

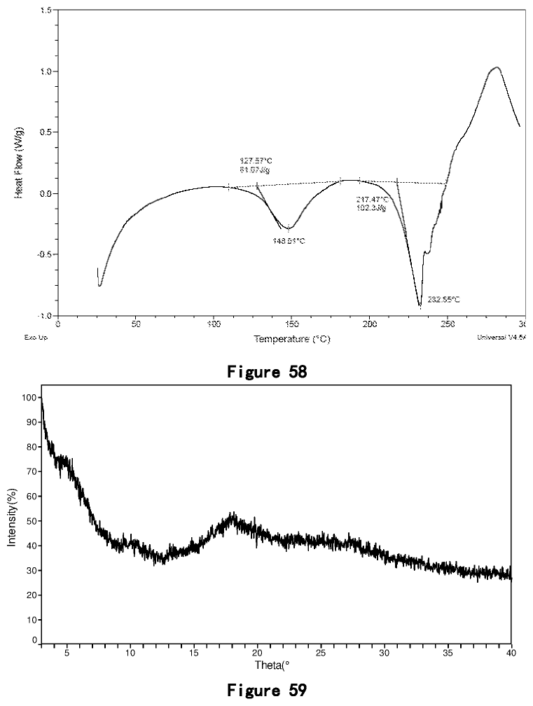

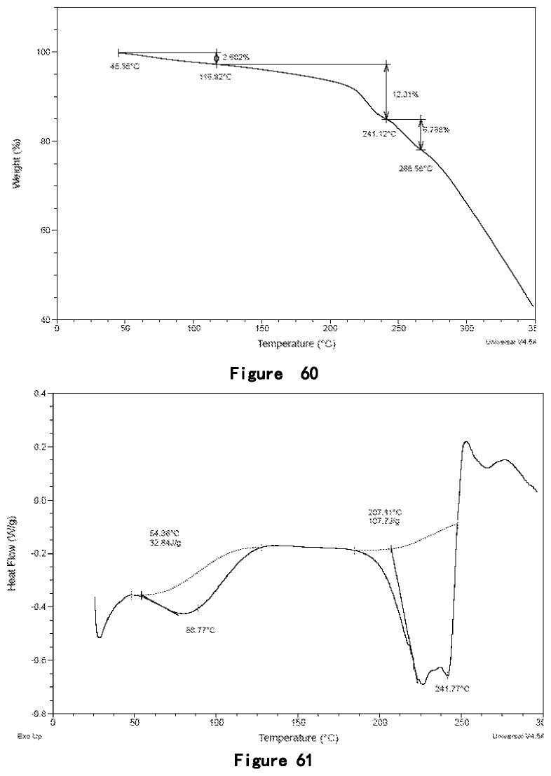

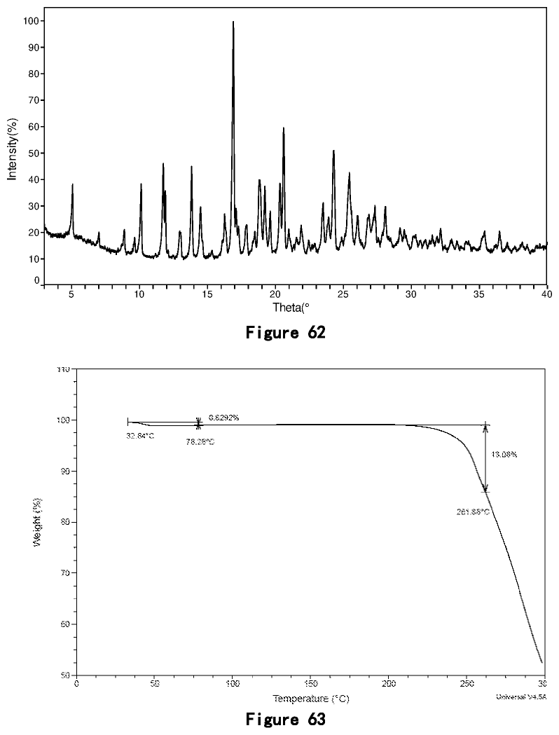

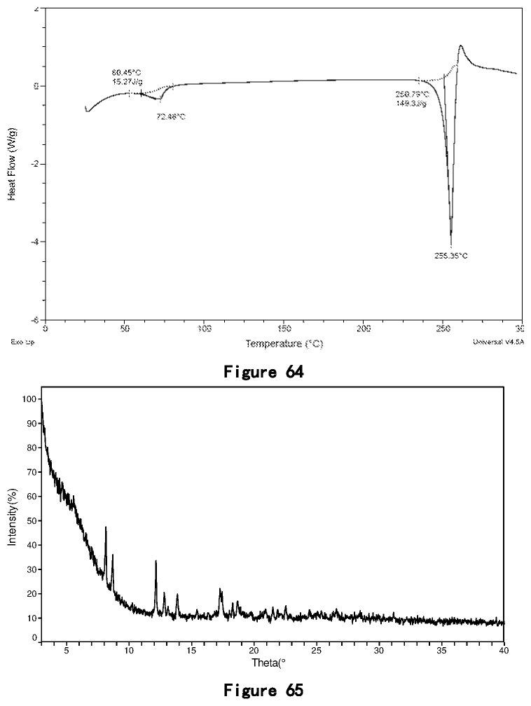

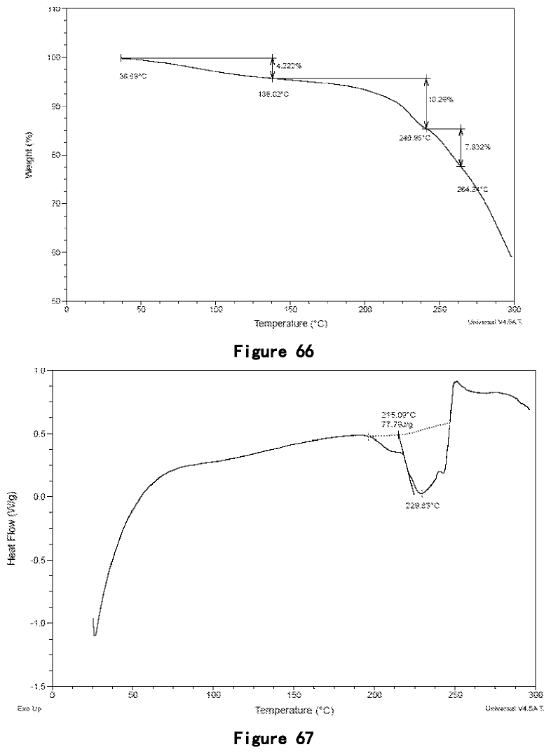

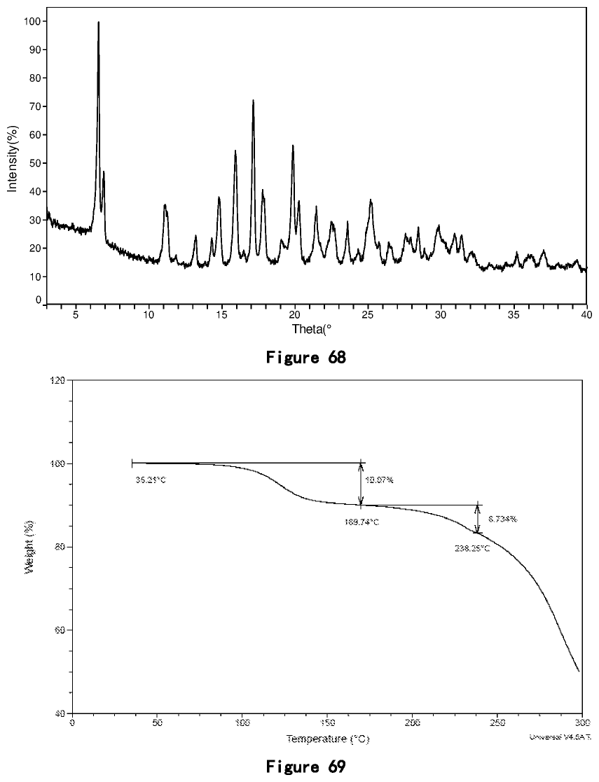

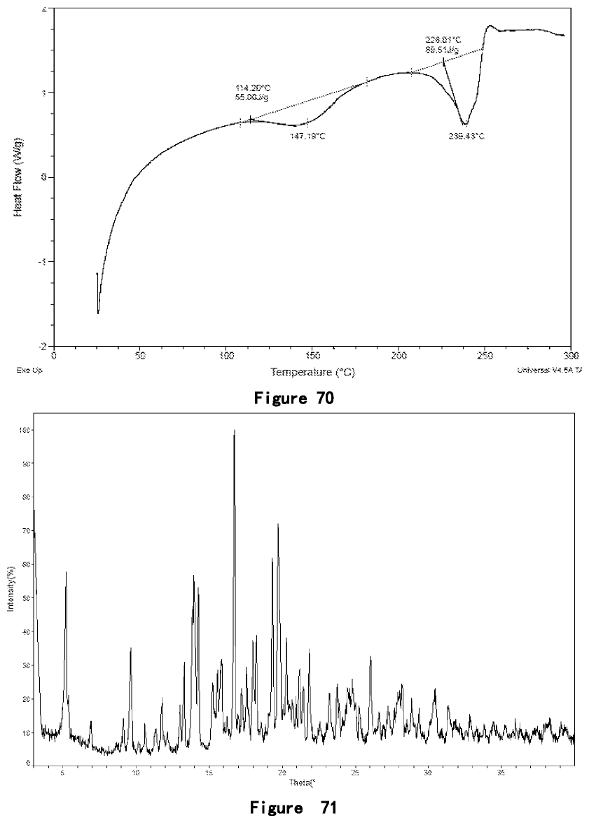

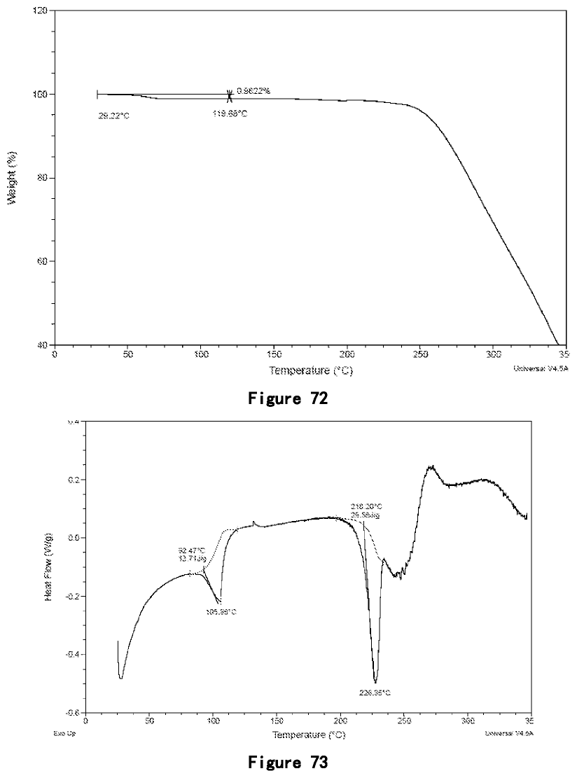

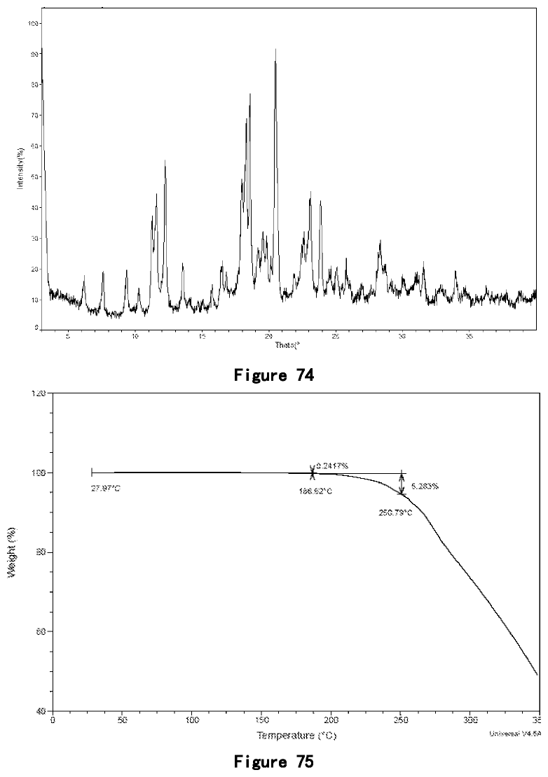

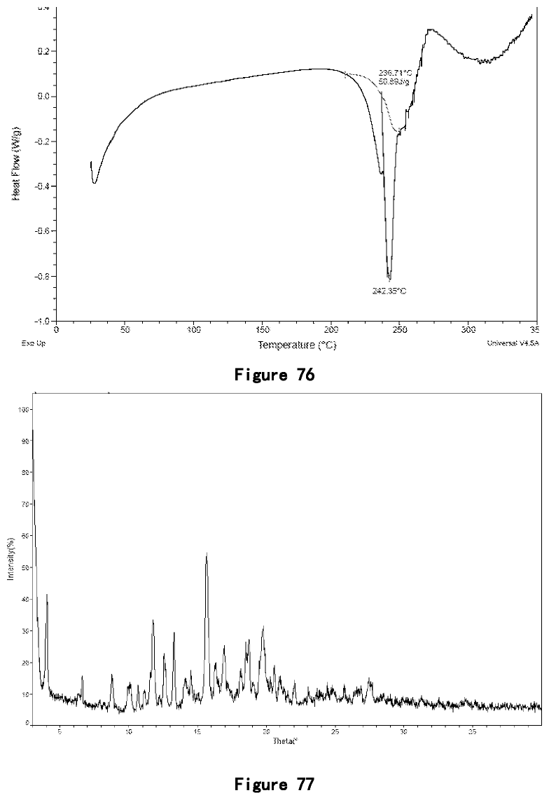

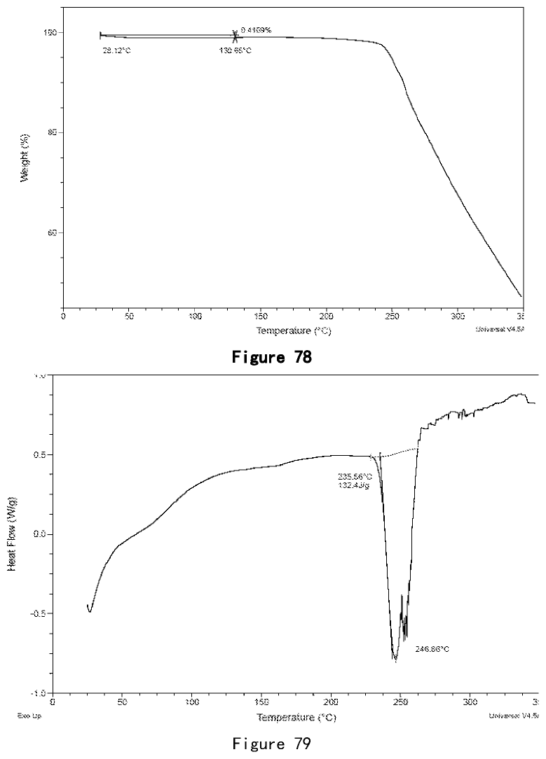

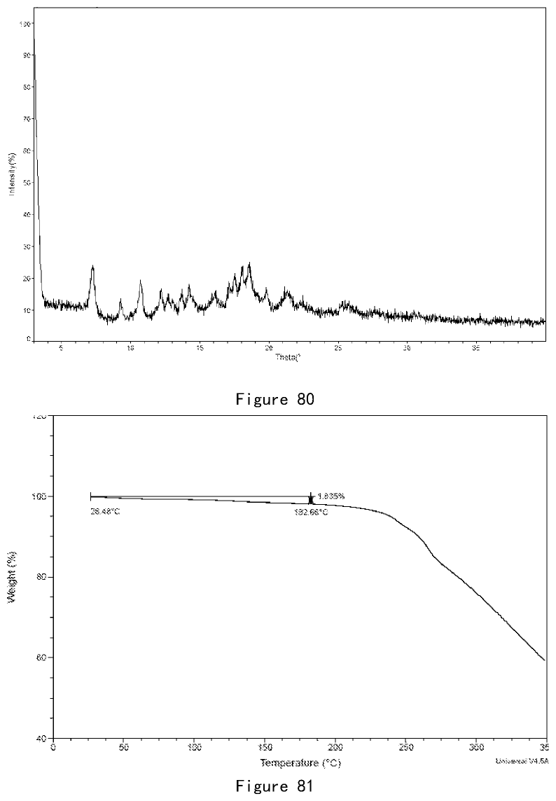

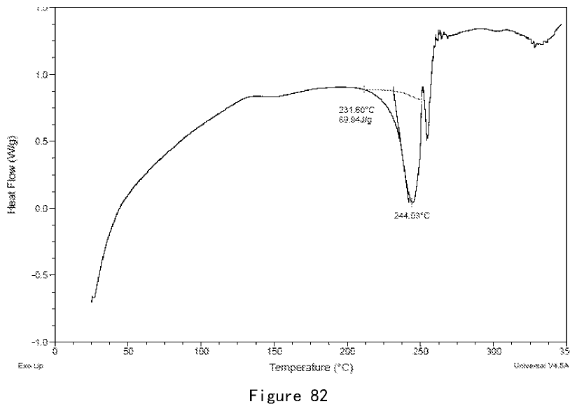

45. The form according to claim 42, further comprising on or more of:
1) the TGA as shown in FIG. 49; and
2) the DSC diagram as shown in FIG. 50.

46.-74. (canceled)

75. A method for preparing an amorphous or crystalline form of the salt of a compound of Formula 1 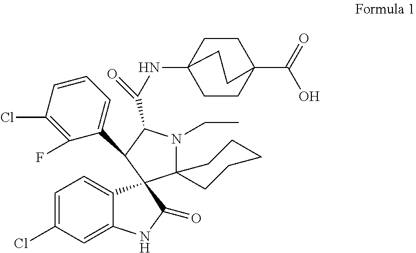
comprising the steps of:
a) reacting the compound of Formula 1 with an acid in an organic solvent;
b) preparing the corresponding amorphous or crystalline form,
and
c) further comprising one or more of the following reaction conditions:
i) the acid is an inorganic acid or an organic acid, the inorganic acid is selected from hydrochloric acid, sulfuric acid, or phosphoric acid; the organic acid is selected from hydrobromic acid, methane sulfonic acid, p-toluenesulfonic acid, maleic acid, L-tartaric acid, fumaric acid, citric acid, malic acid, or succinic acid;
ii) the molar ratio of the compound of Formula 11 to the acid is 1:(1-1.5);
iii) the organic solvent is one or more of alkane solvents, alcohol solvents, ketone solvents, ester solvents, aromatic hydrocarbon solvents, halogenated hydrocarbon solvents, nitrile solvents, ether solvents, aliphatic hydrocarbon solvents, DMF, and DMSO;
iv) the mass-volume ratio of the compound of Formula 1 to the organic solvent is 100 mg: (0.1-1 mL);
v) the reaction temperature is from room temperature to solvent reflux temperature; and
vi) the reaction time is 1 h-36 h.

76. (canceled)

77. A method for preparing an amorphous or crystalline form of the salt of a compound of Formula 1, 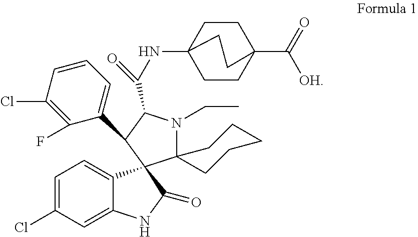
comprising the steps of:
a) mixing the compound of Formula 1 with an organic solvent;
b) adding a mixture of an acid and an organic solvent;
c) stirring;
d) filtering; and
optionally, drying under vacuum at
e) 40-60° C.

78.-80. (canceled)

81. A method for preparing an amorphous or crystalline form of a solvate of the compound of formula 1, 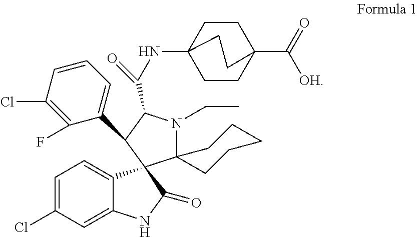
comprising the steps of:
a) contacting or reacting the compound 1 with a solvent;
b) preparing the corresponding amorphous or crystalline form; and
further comprising one or more of the following reaction conditions:
i) the solvent is one or more of water, isopropyl ether, trifluoroethanol, acetonitrile, dimethyl sulfoxide, tetrahydrofuran, ethyl acetate, toluene, and methylcyclohexane;
ii) the mass-volume ratio of the compound of Formula 1 to the solvent is 100 mg: (1-15 mL);
iii) the crystallization temperature is 20-50° C.; and
iv) the crystallization time is 1-48 h.

82. (canceled)

83. A method for preparing an amorphous or crystalline form of a compound of Formula 1, 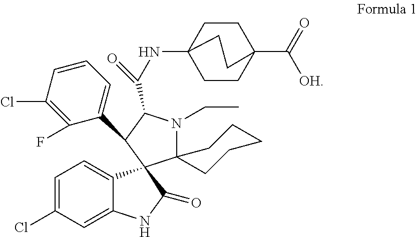
comprising the steps of:
a) contacting or reacting the compound of Formula 1 with a solvent;
preparing a corresponding amorphous or crystalline form,
c) further comprising one or more of the following reaction conditions:
i) the solvent is one or more of water, alkane solvent, alcohol solvent, ketone solvent, ester solvent, aromatic hydrocarbon solvent, halogenated hydrocarbon solvent, nitrile solvent, ether solvent, aliphatic hydrocarbon solvent, acetonitrile, DMF and DMSO;
ii) the mass-volume ratio of the compound 1 to the solvent is 100 mg: (0.1-3 mL);
iii) the crystallization temperature is 20-50° C.; and/or
iv) the crystallization time is 1-48 h.

84. (canceled)

85. A pharmaceutical composition comprising an amorphous or crystalline form of the compound of Formula 1, 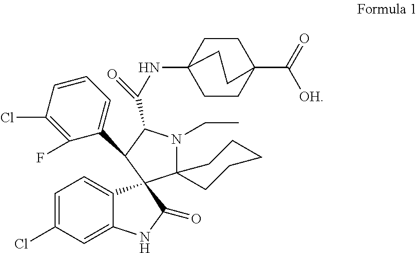
or a salt or solvate thereof comprising a compound form of claim 1 and one or more pharmaceutically acceptable excipients.

86. A method for the treatment of one or more hyperproliferative diseases comprising administering a form of the compound of Formula 1 according to claim 1.

87. The method of claim 86, wherein the hyperproliferative disease is a cancer selected from the group consisting of one or more of adrenal cortical cancer, advanced cancer, anal cancer, aplastic anemia, cholangiocarcinoma, bladder cancer, bone cancer, bone metastasis, adult brain tumor, adult CNS tumor, pediatric brain tumor, pediatric CNS tumor breast cancer, male breast cancer, pediatric cancer, primary cancer of unknown origin, giant lymphadenopathy, cervical cancer, colon cancer, rectal cancer, endometrial cancer, esophageal cancer, Ewing's tumor, eye cancer, gallbladder cancer, gastrointestinal carcinoid tumor, gastrointestinal stromal tumor (GIST), gestational trophoblastic disease, Hodgkin's disease, Kaposi's sarcoma, kidney cancer, laryngeal and hypopharyngeal cancer, adult acute lymphoblastic leukemia (ALL), acute myelogenous leukemia (AML), chronic lymphocytic leukemia (CLL), chronic myelogenous leukemia (CML), chronic myelogenous leukemia (CMML), childhood leukemia, liver cancer, non-small cell lung cancer, small cell lung cancer, lung carcinoid tumor, skin lymphoma, malignant mesothelioma, multiple myeloma, myelodysplastic syndrome, nasal cavity cancer, paranasal sinus cancer, nasopharyngeal carcinoma, neuroblastoma, Non-Hodgkin's lymphoma, pediatric non-Hodgkin's lymphoma, oral cancer, oropharyngeal cancer, osteosarcoma, ovarian cancer, pancreatic cancer, penile cancer, pituitary tumor, prostate cancer, retinoblastoma, rhabdomyosarcoma, salivary gland cancer, Sarcoma soft tissue cancer, basal skin cancer, squamous cell skin cancer, skin cancer-melanoma, small intestine cancer, gastric cancer, testicular cancer, thymus cancer, thyroid cancer, uterine sarcoma, vaginal cancer, vulvar cancer, Waldenster Renal macroglobulinemia, and Welms' tumor.
